(19) United States
(12) Patent Application Publication
(10) Pub. No.: US 2015/0105338 A1
(43) Pub. Date: Apr. 16, 2015

(54) THERAPEUTIC COMPOSITIONS COMPRISING PHENOLIC ACIDS FOR TREATING CONDITIONS RELATED TO INAPPROPRIATE PLATELET AGGREGATION

(71) Applicant: PROVEXIS NATURAL PRODUCTS LIMITED, Windsor (GB)

(72) Inventor: Niamh O'Kennedy, Windsor (GB)

(73) Assignee: PROVEXIS NATURAL PRODUCTS LIMITED, Windsor (GB)

(21) Appl. No.: 14/333,787

(22) Filed: Jul. 17, 2014

Related U.S. Application Data
(63) Continuation of application No. 13/126,137, filed on Apr. 26, 2011, now abandoned.

(30) Foreign Application Priority Data
Oct. 31, 2008 (GB) 0819958.0

(51) Int. Cl. A61K 31/192; A61K 31/353; A61K 31/7048; A61K 47/48

(52) U.S. Cl. CPC A61K 31/192; A61K 47/48092; A61K 31/353; A61K 31/7048
USPC 514/27; 562/475; 514/568; 514/456; 562/476; 514/25; 562/478; 514/570; 562/493

(57) ABSTRACT

The invention provides compositions comprising a therapeutically effective amount of a compound of general formula (I): wherein R1, R2 and R3 may be independently selected from H, OH and OMe; wherein X is C₁ or C₂ and wherein for C2 each carbon is linked by a single or multiple bond (preferably a double bond) and is substituted with one or more H or OH, for use as a medicament for treating or preventing the development of medical conditions characterised by inappropriate platelet aggregation. The compositions of the invention may be used to maintain heart health by reducing platelet aggregation; benefit the circulation; and/or normalize or otherwise benefit blood flow.

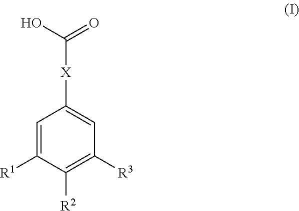

The present invention concerns compositions comprising antithrombotic agents and particularly phenolic compounds that inhibit platelet aggregation.

It is well established that consumption of fruits and vegetables is an important preventative measure by which the risk of cardiovascular diseases can be reduced. Accordingly considerable effort has been expended in an attempt to identify compounds derived from fruits and vegetables that have a role in the prevention of heart disease.

Particular interest has been shown in agents that inhibit platelet aggregation. When platelets aggregate within the circulatory system, thrombi are formed which are large enough to block blood vessels. However before full aggregation takes place, platelets can circulate in an activated condition. When in this state, platelet stickiness is greatly increased, and they can stick to each other, to other blood cells, or to components of the blood such as lipid-rich chylomicrons. This causes micro-aggregates to form, and lowers the fluidity of the blood, affecting blood flow locally, and the circulation systemically. Reducing platelet aggregability helps to maintain the blood in a fluid and low-coagulable state. This helps to normalise blood flow, by preventing micro-aggregates forming within the circulation, and by preventing the adherence of platelets to blood vessel walls or fatty plaques.

In light of the above, it will be appreciated that agents capable of inhibiting platelet aggregation are of use in preventing coronary disease, for example myocardial infarctions and stroke and in preventing further thrombo-embolic events in patients who have suffered myocardial infarction, stroke or unstable angina. In addition, such agents may be of use in preventing restenosis following angioplasty and bypass procedures. Moreover, these agents may be of use in the treatment of coronary disease resulting from thrombo-embolic disorders such as myocardial infarction in conjunction with thrombolytic therapy.

There are many known anti-platelet-aggregation agents that act at different stages of platelet production and action. Aspirin (acetylsalicylic acid) is the most widely used and studied. Dipyridamole and ticlopidine have also been used. Aspirin's antiplatelet activity is due to irreversible inhibition of platelet cycle-oxygenase, thus preventing the synthesis of thromboxane A2, a compound that causes platelet aggregation. Indobufen is a reversible inhibitor of platelet cyclo-oxygenase. Some compounds are direct inhibitors of thromboxane A2 synthase, for example pirmagrel, or act as antagonists at thromboxane receptors, for example sulotroban.

International Patent application WO 99/55350 discloses that water-soluble extracts from a number of fruits exhibit an ability to inhibit platelet aggregation. It was considered surprising that anti-platelet-aggregation activity was found to be water soluble because, in contrast, active extracts known to the art at that time were lipid soluble compounds (e.g. lycopene). These water-soluble extracts were found to have significant efficacy for preventing or reducing platelet aggregation and have been marketed, with Food Standards Agency approval in Europe, as a nutritional supplement with health benefits.

The active component of the WO 99/55350 fruit extract was analysed by mass spectroscopy (MS) and nuclear magnetic-resonance (NMR) spectroscopy and found to contain a mixture of nucleosides having platelet aggregation inhibiting activity.

The present invention is based upon the inventor's realisation that nucleosides, within water-soluble plant extracts, may not be the only compounds within such extracts that prevent anti-platelet aggregation. They therefore exerted considerable effort to further fractionate the water-soluble extracts described in WO 99/55350 in an attempt to identify further compounds that have efficacy for inhibiting platelet aggregation.

It has now been found that water-soluble plant extracts contain a number of compounds that will modulate platelet aggregation (see below). This new knowledge has enabled the inventors to develop new compositions with efficacy for inhibiting platelet aggregation.

Thus according to one aspect of the invention there is provided a composition comprising a therapeutic or prophylactic effective amount of plant phenol or derivative thereof for use as a medicament for treating or preventing the development of medical conditions characterised by inappropriate platelet aggregation.

According to a further aspect of the invention there is provided a composition comprising a therapeutically effective amount of a compound of general formula (I):

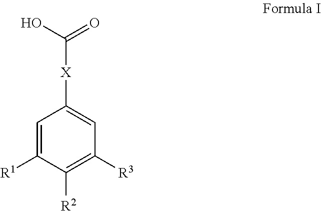

wherein R1, R2 and R3 may be independently selected from H, OH and OMe;

wherein X is C₁ or C₂ and wherein for C₂ each carbon is linked by a single or multiple bond (preferably a double bond) and is substituted with one or more H or OH;

for use as a medicament for treating or preventing the development of medical conditions characterised by inappropriate platelet aggregation.

It is preferred that X comprises either a single saturated carbon or an unsubstituted C═C group. The families of compounds described by these structures are known as benzoic and cinnamic acids, respectively.

It is preferred that R2 is either a hydrogen or an hydroxyl group.

Compositions according to the invention may be used to treat, and in particular prevent the development, of disease states that are characterised by inappropriate platelet aggregation. The inventors have established that the compositions of the invention are particularly useful for:
(a) preventing or reducing the occurrence of a hypercoagulable or prothrombotic state, such as is often associated with conditions such as diabetes mellitus, inflammatory bowel disease, hyperlipidaemia
(b) preventing or reducing the development of atherosclerosis
(c) preventing the development of coronary disease (e.g. myocardial infarctions and stroke and in preventing further thrombo-embolic events in patients who have suffered myocardial infarction, stroke or unstable angina).
(d) preventing the development of restenosis following angioplasty and bypass procedures.
(e) treating coronary disease resulting from thrombo-embolic disorders such as myocardial infarction in conjunction with thrombolytic therapy.
(f) preventing or reducing the risk of deep vein thrombosis
(g) benefiting the circulation to maintain good circulatory health
(h) maintaining healthy blood flow in the cardiovascular system.

It will be appreciated that compositions of the invention will have general health benefits for maintaining cardiovascular and heart health by reducing platelet aggregation, benefiting the circulation, and/or normalizing or otherwise benefiting blood flow (e.g. as outlined in (g) and (h) above).

Indeed so advantageous are these uses of the compositions, that the invention further provides a composition comprising a therapeutically effective amount of a compound of general formula (I):

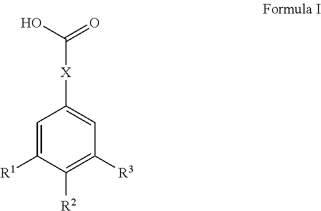

wherein R1, R2 and R3 may be independently selected from H, OH and OMe;

wherein X is C₁ or C₂ and wherein for C₂ each carbon is linked by a single or multiple bond (preferably a double bond) and is substituted with one or more H or OH;
for use as a medicament for normalizing or otherwise benefiting blood flow in a patient.

Compositions will be useful as pharmaceutical products but will also represent beneficial functional foods or “nutraceuticals”. Accordingly preferred uses of the compositions are as medicaments and functional foods or drinks (as outlined below).

The inventors realised that the phenol derivatives of general formula I are useful for preventing platelet aggregation after conducting detailed analysis of the activity of a myriad of compounds contained within fruit extracts (see Example 1).

In particular cinnamic acid, and derivatives thereof, were found to be particularly effective for inhibiting platelet aggregation. Therefore the compositions according to the invention preferably comprise cinnamic acid or a derivative thereof as defined by formula II:

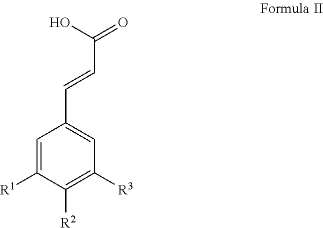

In formula II, R1 and R2 and R3 are as previously defined.

The compound may be Cinnamic acid per se (where R1, R2 and R3 of formula II are H) or may be any one of a number of derivatives, including:

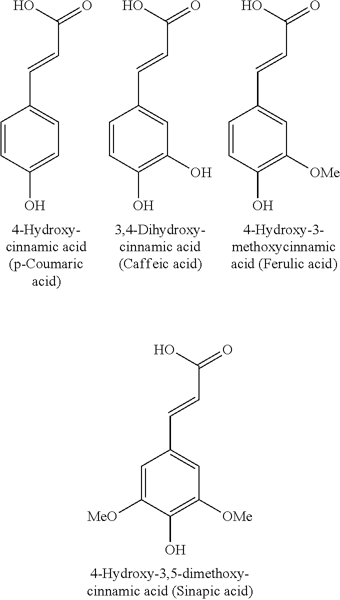

The inventors also established that a further class of plant phenol derivatives, benzoic acids, and derivatives thereof found in fruit, are also effective for inhibiting platelet aggregation. Therefore, in another embodiment of the invention the composition comprises a benzoic acid or derivative thereof as define by formula III:

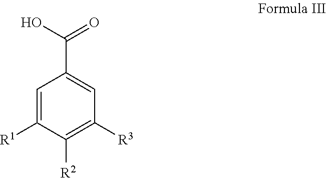

In formula III, R1 and R2 and R3 are as previously defined.

Accordingly preferred compositions according to invention may comprise Benzoic acid per se (wherein each of R1, R2 and R3 are H) or any one of a number of derivatives, for example:

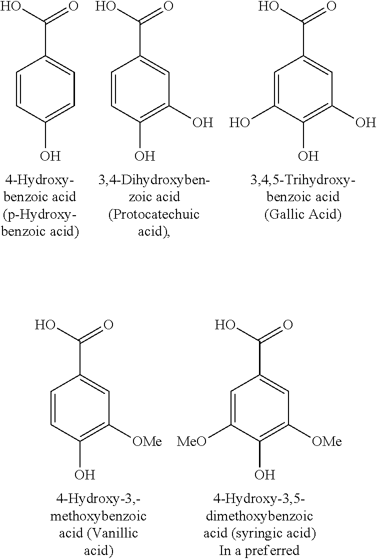

embodiment of the invention the composition comprises at least one compound selected from cinnamic acid and derivatives thereof disclosed above; and at least one compound selected from benzoic acid and derivatives thereof disclosed above. For instance the composition may comprise Caffeic acid and syringic acid. Alternatively the composition may comprise p-Coumaric acid and Gallic Acid.

During the inventors work with fruit extracts they were surprised to discover that compounds of General formula (I), (II) or (III) that are conjugated with other molecules either via an ester linkage at the carboxylic acid group, to form a carboxylic ester, or via an ether linkage at a phenolic hydroxyl substituent, to form a glycoside, are particularly efficacious for reducing platelet aggregation and therefore useful for treating or preventing the development of a variety of cardiovascular conditions. It is therefore preferred that the compositions comprise a compound of General formula (I), (II) or (III) conjugated with other molecules.

It is preferred that the compounds are conjugated to sugars to form glycosides. The sugar is preferably a hexose or pentose sugar or derivatives thereof.

By the term “glycoside” we mean at least one hexose or pentose sugar residue; preferably 1-5 and more preferably 1-3 monosaccharide units are added to the compound by reaction at an OH group on the compound.

Glucose, galactose or arabinose and also de/tri-saccharides of these sugars are most preferably added to the compound to form phenolic acid derivative glycosides.

Alternatively the compounds may be conjugated to a number of compounds found in plants (e.g. tartaric acid, quinic acid) to form esters. Such compounds may be open chain compounds such as tartaric acid, or heterocyclic compounds such as quinic acid and may be derived from the carbohydrate pathway in plants. Tartaric acid or quinic acid are most preferably added to the compound to form phenolic ester derivatives.

It is preferred that the composition comprises a glycoside of the compounds of general formula I, II or III selected from the group comprising: Caffeic acid 3-O-glycoside, Caffeic acid 4-O-glycoside, Ferulic acid 4-O-glycoside, p-Coumaric acid 4-O-glycoside, or an esterified derivative of the compounds of general formula I, II or III selected from the group comprising Caffeoylquinic acids (e.g. 3-O-Caffeoylquinic acid, 4-O-Caffeoylquinic acid or 5-O-Caffeoylquinic acid), Feruloylquinic acids, p-Coumaroylquinic acids, Caffeoyltartaric acids, Feruloyltartaric acids, p-Coumaroyltartaric acids, dimers of quinic acid derivatives.

Preferred compositions may comprise at least one glycoside of Cinnamic acid or derivative thereof selected from the compounds listed above and may also comprise at least one glycoside of a Benzoic acid or derivative thereof selected from the compounds listed above.

Most preferred compositions according to the invention comprise at least one, two, three or all of the following Glycosylated phenolic acid or phenolic esters In the specified amounts:
Caffeic acid glycoside (0.01-1 mg/g);
p-Coumaric acid hexose/dihydrokaempferol hexose mixture (0.05-2.5 mg/g)
Ferulic acid glycoside (0.025-5 mg/g); and/or
p-Coumaric acid derivative (0.01-1 mg/g).

### Other Bioactive Compounds

The inventors also established that flavonoids, and derivatives thereof, and nucleotides/nucleosides derivable from fruit will also inhibit platelet aggregation. Therefore, in preferred embodiments of the invention, the phenolic bioactives may be combined with flavonoid or nucleotide/nucleoside bioactive molecules.

### Flavonoids

In a preferred embodiment the compositions also contain a flavonoid, or derivatives thereof.

In particular the inventors have established that flavonoids of General formulae IV and VI (see below) have activity for modulating platelet aggregation.

The composition preferably contains a flavonoid of general formula (IV):

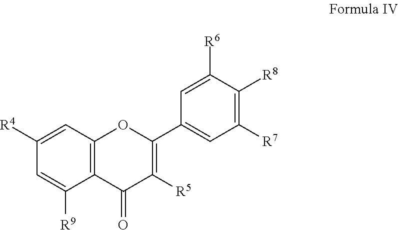

wherein R4, R5, R6, R7, R8 and R9 are independently H, OH.

The inventors have found that flavonoids of formula IV, which have particular antiplatelet activity, have hydroxyl groups at R4, R8 and R9. Accordingly the flavonoid is preferably of general formula (V)

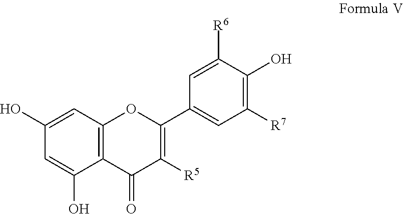

wherein R5, R6 and R7 am independently H, OH

Preferred compounds of general formula IV or V include:

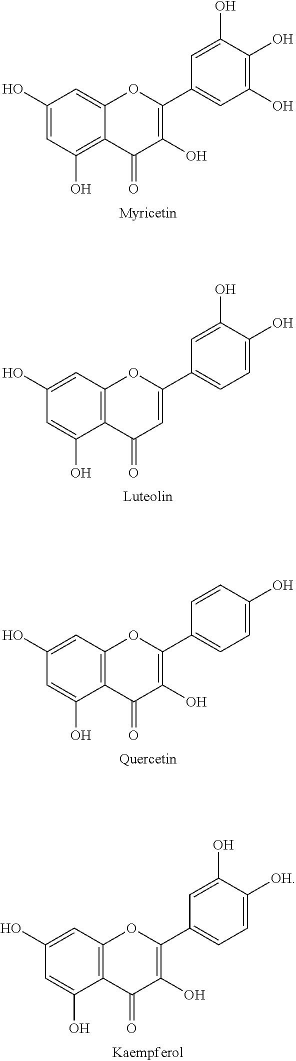

It is most preferred that the composition comprises Quercetin or Kaempferol or deriviatives thereof.

Naringenin and derivatives thereof represent another type of which the inventors have found have activity for inhibiting platelet aggregation. Therefore the composition may comprise molecules of general formula VI.

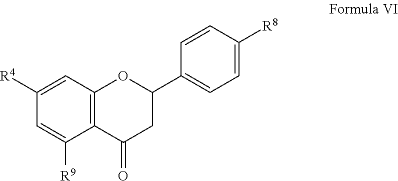

R4, R8 and R9 are as previously defined.

It is more preferred that this type of flavonoid has general formula VII

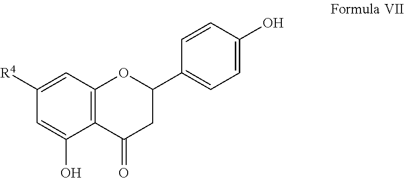

A preferred compound defined by Formula VII that may be used according to the invention is Naringenin

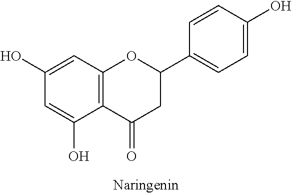 [SMILES: O=C1CC(c2ccc(O)cc2)Oc2cc(O)cc(O)c21]

### Conjugated Flavonoids

The inventors further established that the compounds of General formula (IV)-(VII) that are conjugated with other molecules are particularly efficacious for reducing platelet aggregation. Therefore in a preferred embodiment of the invention, the flavonoid compounds are conjugated with rutin, quinic acid, an amino acid (e.g. tyrosine), anthocyanins (e.g. malvidin or petunidin) or more preferably a glycoside group (as defined above). In a most preferred embodiment of the invention, the composition comprises flavonoid compounds conjugated with the same molecules as defined above for phenolic compounds (i.e. to sugars, tartaric acid, quinic acid and the like).

Most preferred flavonoid compounds with anti-platelet aggregation properties are glycosides of the compounds of general formula V. Specific examples of glycosides of these compounds include:

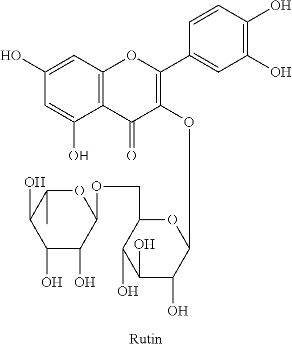

A most preferred glycoslyated flavonoid compound according to general formula (VII) is Naringin.

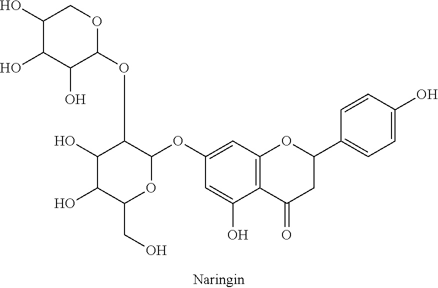

The inventors have found that the phenolic and flavonoid bioactive compounds discussed above may also be conjugated with each other. For example, Caffeic acid 4-O-Rutinoside is a molecule with anti-platelet aggregation properties where a glycoside link is made between Caffeic acid and a sugar residue on Rutin (which comprise Quercetin). Therefore in one embodiment of the invention the composition may comprise a conjugate of a phenolic acid based compound and a flavonoid.

If is most preferred that compositions according to the invention comprise a phenolic bioactive (as discussed above) and the glycosylated flavonoid: Rutin (0.01-1 mg/g).

According a preferred composition comprises:
a) The following Glycosylated phenolic acid or phenolic esters:
Caffeic acid glycoside (0.01-1 mg/g);
p-Coumaric acid hexose/dihydrokaempferol hexose mixture (0.05-2.5 mg/g)
Ferulic acid glycoside (0.025-5 mg/g); and/or
p-Coumaric acid derivative (0.01-1 mg/g); and
(b) The glycosylated flavonoid: Rutin (0.01-1 mg/g).

In a preferred embodiment of the invention the composition may further comprise a nucleoside or nucleotide that may be isolatable from plants.

The inventors have appreciated that such molecules have anti-platelet aggregation activity and are particularly effective when combined with compounds of general formula I.

Examples of nucleosides/nucleotides that the inventors have found to be active include: Adenosine 5′-monophosphate, Cytidine, Uridine, Adenosine, Inosine, Guanosine and Guanosine 5′-monophosphate.

It is preferred that compositions according to the invention further comprise Guanosine (0.1-5 mg/g); and/or Adenosine 3′-monophosphate (0.5-25 mg/g)

Compositions comprising: (i) bioactive compounds of general formula I; (ii) bioactive compounds of general formula IV and/or VI; (iii) and at least one nucleoside or nucleotide represent most preferred compositions that may be used to modulate platelet activity. A most preferred composition therefore comprises
(a) The following Glycosylated phenolic acid or phenolic esters:
Caffeic acid glycoside (0.01-1 mg/g);
p-Coumaric acid hexose/dihydrokaempferol hexose mixture (0.05-2.5 mg/g)
Ferulic acid glycoside (0.025-5 mg/g); and/or
p-Coumaric acid derivative (0.01-1 mg/g); and
(b) The glycosylated flavonoid: Rutin (0.01-1 mg/g); and
(c) Guanosine(0.1-5 mg/g); and/or Adenosine 3′-monophosphate (0.5-25 mg/g)

### Preparation of Bioactive Compounds for Use According to the Invention

It will be appreciated that the bioactive compounds may be synthesised using techniques known to the art of organic chemistry. Phenolic bioactives as-well-as flavonoids and nucleosides/nucleotides can be synthesised.

Phenolic acids may be synthesised following a variety of methods, for example by direct carboxylation of a phenol, carried out by heating the sodium salt under pressure with carbon dioxide (the Kolbe-Schmidt reaction), a system particularly suitable for preparation of mono, di- or trihydroxylated benzoic acids. Alternatively, Grignard reactions may be used to introduce a carboxyl group onto a phenol with varying substitution patterns. Cinnamic acids (α,β-unsaturated acids) can be derived by exploiting condensation reactions between an aromatic aldehyde and an acid anhydride in the presence of sodium or potassium salts, known as Perkin reactions.

Biomimetic reactions have also been developed to yield cinnamic and benzoic acids difficult to synthesise by other means. Hemi-synthetic methods, for example utilising plant derived enzymes, are in widespread use and are particularly useful for derivation of benzoic and cinnamic acid esters. Feruloyl esterase Type A from Aspergillus niger is commonly used for this purpose. Glycosides can be synthesised using the same enzyme, or alternatively using glucosyltransferases, usually UDP-glucose dependent. As an alternative technique, benzoic and cinnamic glycosides may be isolated from plant cell culture supernatants primed with the appropriate natural precursors. Such methods are widely used to obtain mixtures enriched in otherwise difficult to obtain glycosides and derivatives.

Cell culture biosynthetic methods are also widely used for isolation of flavonoid glycosides. Several alternative synthetic methods may also be used to prepare such compounds, e.g. condensation reactions between malonyl Co-A and a hydroxycinnamate Co-A, catalysed by chalcone synthase, to produce chalcones which are then subjected to ring-closure to give a range of flavonoids. Conjugation of flavonoids can be achieved using a range of flavonoid glycosyltransferases in the presence of UDP-sugars, in very much the same way as described for benzoic and hydroxycinnamic acids. Direct esterification can also be achieved in some cases using Knoevengel reaction conditions.

The bioactive compounds in the composition of the invention may preferably be derivable from plants and more preferably fruits and in a preferred embodiment the compounds are derived from a plant.

The bioactive compounds may preferably be derived from fruit selected from the families Solanaceae, Rutaceae, Cucurbitaceae, Rosaceae, Musaceae, Anacardiaceae, Bromeliaceae, Vitaceae, Arecaceae, Ericaceae, Lauraceae, Sterculiaceae and Poaceae.

Examples of Solanaceae include the tomato, for example the English tomato variety. Examples of Rutaceae include the Citrus species such as Citrus paradis (grapefruit), Citrus sinensis (orange), Citrus limon (lemon) and Citrus aurantifolia (lime). Examples of Cucurbitaceae include Cucurnis melo (melon), e. g. the honeydew melon. Examples of Anacardiaceae include Mangifera indica (mango). Examples of Rosaceae include Pyrus malus or Pyrus sylvestris (apple), Pyrus communis (pear), Amygdalus persica or Prunus persica Var. nectarina (nectarine), Prunus armeniaca (apricot), Prunus domestica (plum), Prunus avium (cherry), Prunus persica (peach), Fragaria anannassa (strawberry) and the blackberry. Examples of Bromeliaceae include Ananas sativus (pineapple). Examples of Vitaceae include Vitis vinifera (grape). Examples of Arecaceae include Phoenix dactylifera (date). Examples of Ericaeae include Vaccinium macrocarpum (blueberry). Examples of Lauraceae include Persea gratissima or Persea americana (avocado). Examples of Sterculiaceae include Theobroma cacao (cocoa). Examples of Poaceae include Zea mays (maize), Sorghum vulgare (sorghum), Triticum aestivum (wheat) and Avena sativa (oats).

Particular examples of fruits, that are sources of the compounds according to the invention are the tomato, grapefruit, melon, mango, melon, pineapple, nectarine, strawberry, plum, banana, cranberry, grape, pear, apple, cocoa bean and avocado.

Particular examples of cereals that are sources of the compounds according to the invention are wheat, maize, oats, sorghum, millet and barley.

It is most preferred that the compounds are derivable from or derived from tomatoes.

The compounds may be isolated from fruit by fractionating fruit extracts and then identifying fractions that contain the compounds. Standard techniques such as Mass spectroscopy (MS) and nuclear magnetic resonance (NMR) spectroscopy may be used to isolate fractions containing phenolic compounds (and flavonoids and nucleosides if required). If desired the isolated compound may be purified from the fruit extract to homogeneity and may then be formulated to form a composition according to the invention as described below. However the inventors have found that it is not necessary to purify the compounds to homogeneity and compositions comprising fruit extracts that are enriched in the active compounds represent preferred compositions according to the invention (see below).

In a preferred embodiment of the invention the bioactive compounds of the invention are derivable from a water-soluble fruit extract prepared as described in WO 99/55350. Accordingly the bioactives may be derived from an extract of a fruit (e.g. a tomato) which is (a) substantially heat stable; (b) is colourless or straw-coloured; (c) is water soluble; and (d) consists of components having a molecular weight of less than 1000 daltons.

Preferred methods of isolating bioactive compounds from fruit extracts are discussed in Example 1.

### Preparation of Compositions According to the Invention Comprising Phenolic Bioactives

It will be appreciated that phenolic bioactives may be synthesised using chemical techniques and such compounds may be used to supplement foods or pharmaceutical products. By way of example a phenolic bioactive according to the invention may be synthesised and added to a fruit extract (e.g. as discussed below) which contains other bioactives (e.g. flavonoids, nueclosides and even other phenolic compounds) to make a preferred composition according to the invention.

Compounds that have been synthesised do novo or compounds isolated from plants may be mixed at the correct concentrations and in appropriate molar ratios to form a composition according to the invention (see below). Such compositions may comprise other agents as discussed in the formulation section below.

It is preferred the compositions are derived from the plants discussed above. Such compositions may be prepared by enriching fruit extracts to maintain or enhance the concentration of the phenolic bioactive compounds and other preferred bioactive compounds described above.

The inventors have found that fruits of the Solanaceae family, may be processed in ways that result in water-soluble extracts that have an optimised phenolic bioactive content. Thus according to a further aspect of the invention there is provided a method of making an extract of fruit of the Solanaceae family wherein fruit is processed to optimise the content of phenolic compounds with activity for inhibiting platelet aggregation comprising the steps of:
(a) Preparing a start mix of homogenised fruit;
(b) Separating a water soluble fraction from fruit solids;
(c) filtration of the water soluble fraction; and
(d) concentration of active agents in the filtration permeate

### (a) Preparing a Start Mix

The flesh of whole fruit, preferably tomatoes, is homogenised, with or without the skin of the fruit to form a paste.

Alternatively, commercially available tomato pastes may be used as the starting material for the preparation of the start mix. Where the starting material for the preparation of the extracts is a tomato paste, if is preferably one that has been produced by means of a “cold-break” process rather than a “hot-break” process. The terms “cold-break” and “hot-break” are well known in the field of tomato processing and commercially available tomato pastes are typically sold as either hot-break or cold-break pastes. Cold-break pastes can be prepared by a process involving homogenisation of the tomato followed by a thermal processing step in which the tomatoes are heated to temperatures of no more than about 60° C., in contrast to hot-break pastes where the homogenised tomatoes are subjected to thermal processing at temperatures of about 95° C., see for example, Anthon et al., J. Agric. Food Chem. 2002, 50, 6153-6159.

The thickness of such pastes (whether from fresh fruit or a commercially available paste) should be adjusted by diluting with water or an aqueous solution (preferably demineralised water) to form a “start mix”. The inventors have found that optimal activity is achieved in the final fruit extract if the start mix is diluted such that it contains less than 33% solids and more preferably less than 20% solids. In one preferred embodiment of the invention the start mix comprises between about 10 and 15% solids (e.g. 13% solids).

The inventors have found that the holding temperature of the start mix can have a significant effect on the activity of the extract. It is therefore preferred that the holding temperature does not exceed 35° C. and more preferably does not exceed 30° C.

The inventors have also found that the pH of the start mix also impacts on the activity of the extract prepared according to the method of the invention. The pH of the mix should be acidic; preferably less than pH 5.5 and in a preferred embodiment the pH should not rise above 4.2. Adjustments to pHs if required, may be made by addition of citric acid.

Furthermore the inventors have found that the browning index of the start mix should also be controlled to optimise activity of the finial extract. Accordingly the browning index of the start mix, defined as the absorbance of the soluble portion at 420 nm, preferably does not exceed 0.4 AU at 4% solids. Browning index is an index of visible browning caused by formation of melanoidins (polymeric conjugates of variable composition, based on sugars and amino acids) and may be measured by centrifuging a 50 mL sample of the start mix at 3500 rpm for 10 minutes at room temperature, removing a portion of the supernatant, diluting it to 4% solids as measured by refractometer, and measuring the absorbance of this solution at 420 nm in a spectrophotometer.

The inventors have found that fruit extracts according to the method of the invention have improved anti-aggregation activity if at least one of the temperature, pH and browning index are controlled in the start mix as discussed above. It is preferred that at least two of these control steps (e.g. temperature and pH; or temperature and browning index) are controlled and more preferred that the temperature, pH and Browning index are controlled as discussed above.

It is most preferred that the start mix is maintained at a temperature that is no higher than 30° C.; at a pH of less than 4.2 and with a browning index that does not exceed 0.4 AU.

### (b) Separating a Water Soluble Fraction From Fruit Solids.

Water-insoluble solids may be removed from a water soluble fraction by using a number of standard techniques.

It is preferred that this step in the methodology removes large-sized (i.e. particle size >500 μ) water insoluble solids from the start mix.

Such solids may be removed by use of:
(a) a decanter (e.g. a Westfalia GEA decanter);
(b) a centrifugal separation step (e.g. a rotating disc centrifuge); or
(c) a separator containing size-adjustable nozzles (e.g. a Westfalia MSB-15 separator, using a mixture of blanks and nozzles sized 0.45).

Alternatively the solids may be allowed to settle and the water soluble fraction simply decanted manually.

Whichever method is used, the inventors have found that for retention of optimal bioactivity in the water-soluble fraction, the operating temperatures should not exceed 60° C. Furthermore it is preferred that the flow rate through the equipment must be such that exposure to this 60° C. temperature does not occur for longer than 60 seconds.

The resulting water-soluble fraction should ideally be cooled after the separation step. When the fraction is to be stored it is preferred that, following separation, it is immediately cooled to <8° C.

In preferred embodiments of step (c) of the method of the invention a decanter may be used, with running temperatures of 40-45° C.

Optionally the separation step may be followed by a second clarification step (e.g. using an Alfa Lavaal Clarifier) to produce a clarified water soluble fraction where all remaining insoluble material has a particle size <500 μ and spin-down solids (i.e. material which is visibly precipitated by centrifugation at 3500 rpm for 10 minutes at room temperature) comprise <1% of the fraction by volume.

The inventors have found that the final product retains the maximum active component concentration if the clarified fraction (however produced) contains less than 10% total solids and more preferably about 8% solids or less.

### (c) Filtration of the Water Soluble Fraction

To remove very fine particulate matter (<500μ) (e.g. protein and large polymeric material such as some pectins), the water soluble fraction should then be filtered and the permeate retained.

Filtration may be accomplished in a single stage, or in a series of filtration steps, starting with a relatively coarse filtration step to remove larger particles of tomato skin and/or other water-insoluble fragments of tomato flesh. Further filtration steps may then be effected to give a substantially clear solution, e.g. a solution that will pass through a 0.2μ filter without loss of solids.

In a preferred embodiment step (c) of the method of the invention comprises a microfiltration step using a filtration unit with ceramic membrane filters (e.g. a Tetra Alcross cross-filtration MF unit equipped with ceramic membrane filters (e.g. Pall Membralox P19-30 multi-element units)). Spiral-wound membranes may also be used as an alternative to ceramic membranes.

Ultrafiltration may also be used as an alternative to microfiltration. A range of pore sizes is acceptable, e.g. 1.4μ, 0.1μ; but the inventors have found that maximum enrichment of the filtration permeate with bioactive components (i.e. minimum losses of bioactive components and maximum exclusion of non-bioactive components) occurs when pore sizes of 0.1 μ are used.

In order to retain optimal bioactivity, temperatures should not rise above 35° C. during this filtration step, and the filtration permeate should be immediately cooled to <8° C. after exiting the filtration membrane. The browning index of the final permeate should not exceed 0.4 AU.

The inventors have found that maximum recovery of bioactive components, and enrichment of the filtration permeate in bioactive components (relative to the unfiltered material), occurs when the starting unfiltered material contains <10% solids, and when the final permeate contains approximately 7% solids and has a browning index <0.4 AU.

Removal of the solids according to steps (a) to (c) has the effect of removing fragments of skin and seeds, large molecular weight proteins and pectins, and carotenoids such as lycopene/other lipids which are stabilised in droplets within the aqueous solution by the presence of pectins and proteins. Thus, the methods provide ways of preparing tomato extracts that are water soluble extracts and are also substantially free of lycopene.

The methods described, in particular the careful control of the length of exposure to temperatures >35° C. (preferably >30° C.), also ensure that the lycopene-free water soluble extracts prepared have not been subject to degradative chemical reactions which result in the production of visible browning (Maillard reactions), as demonstrated by the browning index value of <0.4 AU. This ensures that the formation of amino acid—sugar complexes and melanoidin polymers, which can sequester some of the bioactive components, are kept to a minimum. Thus the methods described result in extracts which are optimised for bioactive component content.

In one preferred embodiment of the method of the invention, the tomato extract is a water soluble extract substantially free of lycopene and capable of passing through a 0.2μ filter without loss of solids, and with a browning index value <0.4 AU.

### (d) Concentration of Active Agents in the Filtration Permeate

The aqueous filtrate is then subjected to further concentration/fractionation steps to provide a bioactive concentrate containing compounds responsible for inhibiting platelet aggregation.

After much experimentation the inventors established that the concentration steps required careful control if peak bioactivity of the final extract was to be retained or enrichment of bioactives is to be achieved in the final concentrated product. The reason for this was found to be, that the progress of heat- and pH-dependent degradative reactions is accelerated as solids concentration increases. They therefore realised that temperature control, and length of exposure to temperature, was more crucial for concentrated extracts than for dilute extracts.

Several methods may be used to concentrate/enrich the water soluble material—provided that the temperature of the extract is not allowed to rise such that degradation of active agents within the extract is not allowed to rise above about 80° C. for dilute fractions and below 40° C. for more concentrated samples.

### Concentration Using Evaporation Techniques

Evaporation of the solution under reduced pressure may be used, under conditions where temperatures do not exceed 60° C.

Preferably, a multi-effect evaporator is used, so that temperatures can be lowered as the liquid passes through the evaporator, ensuring that the more concentrated material is not exposed to temperatures >40° C. whereas the more dilute material can tolerate temperatures of up to 60° C.

Using evaporation, the water soluble extract can be concentrated up to 70% solids, e.g. to 20% solids, or to 50% solids, or to 65% solids. In a most preferred embodiment the final extract comprises 60-62% solids after concentration according to step (d).

The effect of temperature can be quantified by measuring the browning index. Temperatures should be sufficiently low such that the final concentrated product should not exceed 0.8 AU.

The final concentrate formed following steps (a), (b), (c) and utilising evaporator according to step (d) preferably has a browning index of <0.8 AU, a pH of 4.0-4.3 and a density of 1.15-1.20.

### Concentration Using Membrane Processes

Alternatively Membrane processes which allow water to pass through the membrane while retaining all other components within the membrane can also be used. Examples of specific techniques are reverse osmosis, or nanofiltration. Both can be used to concentrate the water soluble extract to the required degree, while operating at low temperatures (<40° C.).

### Drying Techniques

Drying technologies can also be used to remove water from the water-soluble extract. Suitable drying techniques include spray drying, with or without carrier materials (e.g. potato starch, tapioca starch, maltodextrins); vacuum drum drying, with or without carrier materials; or roller drying, with or without carrier materials.

### Preparation of Low Sugar Fruit Extracts Enriched in Phenolic Bioactives

The methods described above were designed for the production of a concentrate containing ail the elements originally present in the water soluble extract (but with optimization of the bioactivity of the compounds defined according to the first aspect of the invention).

In a preferred embodiment of the invention the method of the invention may be adapted to result in a concentrate that is enriched (e.g. 25-35 times) in the bioactive components.

Enrichment of the bioactive components within the water soluble extract can be achieved by removing the soluble sugars which form the largest portion of its dry matter content.

Low sugar fruit extracts may be prepared by following steps (a), (b) and (c) above and then employing a further step in the methods before the final concentration step ((d) above)

Removal of the soluble sugars can be achieved by:
(1) precipitation, e.g. by adding ethanol to the solution to a final concentration of 90%, which will result in precipitation of free glucose, fructose and sucrose;
(2) Partial removal of free sugars by digestion, by enzymes (e.g. glucose oxidase);
(3) by microbial (bacteria or yeast) treatment; or
(4) removing free sugars from the water soluble extract by resin-mediated separation of the extract components

It is preferred that free sugars are removed from the water soluble extract by resin-mediated separation of the extract components ((4) above). The inventors have developed a method in which a food grade resin (Amberlite FPX66) is employed to adsorb all the extract components, with the exception of free sugars, organic acids, and salts. These are not adsorbed by the resin and may be discarded after passing through. The extract components adsorbed onto the resin, which comprise amino acids, bioactive components, and products of browning reactions (Maillard degradation products), are then recovered from the resin by elution with ethanol/water mixtures, e.g. 50% ethanol, or 80% ethanol. Ethanol may be removed from the resulting solution by evaporation under reduced pressure (e.g. in an explosion-proof conventional evaporator, or in a Centritherm centrifugal concentrator), or by reverse osmosis.

After the removal of the sugars the concentration of the product may be adjusted employing the procedures discussed in step (d) above.

The resulting low sugar extract is preferably a concentrated aqueous solution containing <1% sugar, and containing >95% of the bioactive components contained in the start mix.

Preferred methods for preparing fruit extracts enriched in phenolic bioactives according to the invention are disclosed in FIGS. 2 and 3. The amount of phenolic compounds and other bioactives found in these extracts are outlined in Table 1

TABLE 1
bioactive compounds in a tomato extract enriched in phenolic bioactives

| | | | Preferred | | | Preferred | | |
|---|---|---|---|---|---|---|---|---|
| | | | Extract prepared | | | Low Sugar Extract | | |
| | | | according to methods | | | prepared according to | | |
| | | | of Example 2 | | | methods of Example 3 | | |
| | | | lower | upper | | lower | upper | |
| | Compound | Bioactive | range | range | average | range | range | average |
| Group | ID | Compound | mg/g | mg/g | mg/g | mg/g | mg/g | mg/g |
| Nucleosides | 1 | Cytidine | 0.487 | 2.051 | 1.709 | 21.971 | 36.911 | 30.759 |
| | 2 | Adenosine | 0.382 | 2.440 | 2.033 | 1.800 | 2.927 | 2.439 |
| | 3 | Uridine | 0.414 | 2.089 | 1.741 | 21.917 | 31.340 | 26.117 |
| | 4 | Guanosine | 0.400 | 1.759 | 1.466 | 6.970 | 19.354 | 16.128 |
| Nucleotides | 5 | Adenosine 3′- | 1.312 | 11.491 | 9.576 | 6.421 | 16.087 | 13.406 |
| | | monophospate | | | | | | |
| | 6 | Adenosine 5′- | | | | | | |
| | | monophospate | | | | | | |
| Phenolic | 7 | Mixed phenolic acid | 0.352 | 0.956 | 0.796 | 20.982 | 145.537 | 121.281 |
| acid | | glycosides | | | | | | |
| glycosides | 8 | p-Coumaric acid | 0.050 | 0.456 | 0.380 | 9.418 | 11.867 | 9.889 |
| | | hexose/quinic acid | | | | | | |
| | | derivative | | | | | | |
| | 9 | Caffeic acid | 0.069 | 0.477 | 0.398 | 3.736 | 13.402 | 11.168 |
| | | glucoside | | | | | | |
| | 10 | Ferulic acid hexose | 0.028 | 0.048 | 0.040 | 0.706 | 1.340 | 1.117 |
| | 11 | p-Coumaric acid | 0.277 | 0.997 | 0.831 | 26.121 | 40.288 | 33.573 |
| | | hexose/ | | | | | | |
| | | dihydrokaempferol | | | | | | |
| | | hexose mixture | | | | | | |
| | 12 | p-Coumaric acid/ | 0.170 | 1.419 | 1.182 | 90.872 | 131.722 | 109.768 |
| | | caffeic acid | | | | | | |
| | | conjugate, | | | | | | |
| | | glycosylated | | | | | | |
| | 13 | Ferulic acid | 0.155 | 1.199 | 0.999 | 85.333 | 199.679 | 166.399 |
| | | glycoside | | | | | | |
| | 14 | Chlorogenic acid | 0.131 | 0.953 | 0.794 | 18.274 | 43.366 | 36.138 |
| Phenolic | 15 | p-Coumaric acid | 0.105 | 0.332 | 0.277 | 8.620 | 16.584 | 13.820 |
| ester | | derivative | | | | | | |
| derivatives | 16 | Caffeoyl-quinic acid | 0.066 | 0.701 | 0.584 | 13.850 | 85.176 | 70.980 |
| | | dimer #1 | | | | | | |
| | 17 | Caffeoyl-quinic acid | 0.142 | 0.701 | 0.584 | 12.672 | 22.731 | 18.943 |
| | | dimer #2 | | | | | | |
| Phenolic | 18 | Caffeic acid | 0.058 | 0.873 | 0.727 | 5.842 | 9.042 | 7.535 |
| acids | 19 | p-coumaric acid | 0.046 | 0.488 | 0.407 | 11.403 | 27.568 | 22.974 |
| | 20 | Benzoic acid | 0.006 | 0.077 | 0.064 | 0.959 | 1.554 | 1.295 |
| | 21 | Ferulic acid | 0.016 | 0.140 | 0.117 | 0.584 | 1.113 | 0.927 |
| | 22 | Cinnamic acid | 0.028 | 0.084 | 0.070 | 1.966 | 6.896 | 5.747 |
| Flavonoid | 23 | Quercetin-3-O- | 0.050 | 0.324 | 0.270 | 8.463 | 13.257 | 11.048 |
| glycosides | | glycoside | | | | | | |
| | 24 | Kaempferol | 0.008 | 0.049 | 0.041 | 1.269 | 5.277 | 4.398 |
| | | glycoside | | | | | | |
| | 25 | Quercetin-3-O- | 0.157 | 0.610 | 0.508 | 14.679 | 24.799 | 20.666 |
| | | trisaccharides | | | | | | |
| | 26 | Naringin | 0.739 | 2.103 | 1.753 | 38.016 | 61.709 | 51.424 |
| | 27 | Rutin | 0.583 | 2.804 | 2.337 | 50.688 | 106.147 | 88.456 |
| Flavonoid | 28 | Flavonoid conjugate | 0.004 | 0.032 | 0.027 | 0.846 | 1.733 | 1.444 |
| ester | 29 | Trace flavonoids + | 1.253 | 3.900 | 3.250 | 90.660 | 319.469 | 266.224 |
| derivatives | | glycosides | | | | | | |
| Flavonoids | 30 | Quercetin | 0.014 | 0.130 | 0.108 | 3.787 | 20.578 | 17.149 |
| | 31 | Kaempferol | 0.039 | 0.180 | 0.150 | 3.749 | 8.230 | 6.858 |
| | 32 | Naringenin | trace | 1.540 | trace | trace | 25.600 | trace |

### Pharmaceutical and Nutraceutical Formulations

The compositions of the invention may be formulated for oral administration. As such, they can be formulated as solutions, suspensions, syrups, tablets, capsules, lozenges and snack bars, inserts and patches by way of example.

Such formulations can be prepared in accordance with methods well known to the art.

For example, the composition may be formed into a syrup or other solution for administration orally, for example as a health drink. One or more excipients selected from sugars, vitamins, flavouring agents, colouring agents, preservatives and thickeners may be included in such syrups or solutions. Tonicity adjusting agents such as sodium chloride, or sugars, can be added to provide a solution of a particular osmotic strength, for example an isotonic solution. One or more pH-adjusting agents, such as buffering agents can also be used to adjust the pH to a particular value, and preferably maintain it at that value. Examples of buffering agents include sodium citrate/citric acid buffers and phosphate buffers.

Alternatively, the composition may be dried (e.g. by spray drying or freeze drying) and the dried product formulated in a solid or semi solid dosage form, for example as a tablet, lozenge, capsule, powder, granulate or gel.

Compositions can be prepared without any additional components. Alternatively, they may be prepared by adsorbing on to a solid support; for example a sugar such as sucrose, lactose, glucose, fructose, mannose or a sugar alcohol such as xylitol, sorbitol or mannitol; or a cellulose derivative. Other particularly useful adsorbents include starch-based adsorbents such as cereal flours for example wheat flour and corn flour.

For tablet formation, the composition is typically mixed with a diluent such as a sugar, e.g. sucrose and lactose, and sugar alcohols such as xylitol, sorbitol and mannitol; or modified cellulose or cellulose derivative such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener can be added, for example ammonium glycyrrhizinate or an artificial sweetener such as aspartame, or sodium saccharinate.

Compositions can also be formulated as powders, granules or semisolids for incorporation into capsules. When used in the form of powders, the extracts can be formulated together with any one or more of the excipients defined above in relation to tablets, or can be presented in an undiluted form. For presentation in the form of a semisolid, the dried extracts can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cod liver oil, herring oil, etc. Such extracts can be filled into capsules of either the hard gelatine or soft gelatine type or made from hard or soft gelatine equivalents, soft gelatine or gelatine-equivalent capsules being preferred for viscous liquid or semisolid fillings.

Compositions according to the invention can also be provided in a powder form for incorporation in to snack food bars for example fruit bars, nut bars, and cereal bars. For presentation in the form of snack food bars, the compositions can be admixed with any one or more ingredients selected from dried fruits such as sun-dried tomatoes, raisins and sultanas, groundnuts or cereals such as oats and wheat.

Compositions according to the invention may also be provided in a powder form for reconstitution as a solution. As such they can also contain soluble excipients such as sugars, buffering agents such as citrate and phosphate buffers, and effervescent agents formed from carbonates, e.g. bicarbonates such as sodium or ammonium bicarbonate, and a solid acid, for example citric acid or an acid citrate salt.

In one preferred embodiment, a composition according to the invention is provided in powder form optionally together with a preferred solid (e.g. powdered) excipient for incorporation into capsules, for example a hard gelatine capsule.

A solid or semisolid dosage form of the present invention can contain up to about 1000 mg of the composition, for example up to about 800 mg.

The composition can be presented as food supplements or food additives, or can be incorporated into foods, for example functional foods or nutraceuticals.

The compositions of the invention can be presented in the form of unit dosage forms containing a defined concentration of compounds with activity for inhibiting platelet aggregation. Such unit dosage forms can be selected so as to achieve a desired level of biological activity. For example, a unit dosage form can contain an amount of up to 1000 mg (dry weight) of a composition according to the present invention, more typically up to 800 mg, for example 50 mg to 800 mg, e.g. 100 mg to 500 mg. Particular amounts of the composition that may be included in a unit dosage form may be selected from 50 mg, 100 mg, 150 mg, 200 mg, 250 mg, 300 mg, 350 mg, 400 mg, 450 mg, 550 mg, 600 mg, 650 mg, 700 mg, 750 mg and 800 mg.

The compositions of the invention can be included in a container, pack or dispenser together with instructions for administration.

### Dosing

For the treatment of the diseases and conditions concerned, the quantity of the active compound or composition according to the invention administered to a patient per day will depend upon the strength of the active compound, the particular condition or disease under treatment and its severity, and ultimately it will be at the discretion of the physician. The amount administered however will typically be a non-toxic amount effective to treat the condition in question.

A sufficient amount of a composition or extract according to the invention should be administered to a subject to achieve a blood concentration of 0.1-50 mg/L total bioactive components over a timecourse of 1.5-3 hours; preferably a blood concentration of 1-5 mg/L total bioactive components over a timecourse of 1.5-3 hours is achieved; and most preferably a blood concentration of about 2.58 mg/L total bioactive components over a timecourse of 1.5-3 hours is achieved.

By way of example for Individual bioactive compounds:

When Caffeic acid (compound 18) is used alone, it should be administered to a subject to achieve a blood concentration of 1-250 mg/L over a timecourse of 1.5-3 hours; preferably a blood concentration of 10-100 mg/L over a timecourse of 1.5-3 hours is achieved; and most preferably a blood concentration of about 57 mg/L over a timecourse of 1.5-3 hours is achieved.

When Caffeic acid glucoside (compound 9) is used alone, it should be administered to a subject to achieve a blood concentration of 0.25-150 mg/L over a timecourse of 1.5-3 hours; preferably a blood concentration of 0.5-75 mg/L over a timecourse of 1.5-3 hours is achieved; and most preferably a blood concentration of about 31 mg/L over a timecourse of 1.5-3 hours is achieved.

When the phenolic acid composition includes a flavonoid or deriviative thereof, the composition may comprise sufficient quercetin (compound 30) to achieve a blood concentration of 0.25-100 mg/L over a timecourse of 1.5-3 hours; preferably a blood concentration of 0.5-50 mg/L over a timecourse of 1.5-3 hours is achieved; and most preferably a blood concentration of about 8 mg/L over a timecourse of 1.5-3 hours is achieved. Alternatively the composition may comprise quercetin-3-O-glycoside (Compound 23) to achieve a blood concentration of 0.25-250 mg/L over a timecourse of 1.5-3 hours: preferably a blood concentration of 1-100 mg/L over a timecourse of 1.5-3 hours is achieved; and most preferably a blood concentration of about 21 mg/L over a timecourse of 1.5-3 hours is achieved.

It will be appreciated that preferred compositions may also comprise a nucleoside or nucleotide. Such compositions may comprise a sufficient amount of the nucleoside cytidine (compound 1) to achieve a blood concentration of 10-1,000 mg/L over a timecourse of 1.5-3 hours; preferably a blood concentration of 25-500 mg/L over a timecourse of 1.5-3 hours is achieved; and most preferably a blood concentration of about 134 mg/L over a timecourse of 1.5-3 hours is achieved. Alternatively the composition may comprise a sufficient amount of the nucleotide AMP to achieve a blood concentration of 75-7500 mg/L over a timecourse of 1.5-3 hours; preferably a blood concentration of 250-1500 mg/L over a timecourse of 1.5-3 hours is achieved; and most preferably a blood concentration of about 749 mg/L over a timecourse of 1.5-3 hours is achieved.

The amount of composition administered to a patient typically will vary according to the concentration of the active compound or ingredients in the compound. However, a typical daily dosage regime for a human patient suffering from a cardiovascular disease may be from 0.0001 to 0.1, preferably 0.001 to 0.05 gram per kilogram body weight of a fruit extract. It will be appreciated by a skilled person that the amount required for specific bioactives can be calculated on the basis of IC50 values (see the Examples) and on the basis of desired blood concentrations (see above)

The compositions can be administered in single or multiple dosage units per day, for example from one to four times daily, preferably one or two times daily.

The compositions of the invention can be administered in solid, liquid or semi-solid form. For example, the extracts can be administered in the form of tomato juice or concentrates thereof alone or in admixture with other fruit juices such as orange juice.

### Indications of Therapeutic Effectiveness

The ability of compositions of the invention to provide beneficial therapeutic effects may be assessed with reference to a number of different parameters. The Examples below provide details of suitable protocols for the assessment of platelet aggregation or primary haemostasis, either of which may be investigated in order to evaluate therapeutic effectiveness. The PFA-100® platelet function analyzer described in the Examples is a relatively new device for the assessment of primary haemostasis, but has been well validated (see, for instance, “The platelet-function analyzer (PFA-100®) for evaluating primary hemostasis” by M. Franchini Hematology, Volume 10, Issue 3 June 2005, pages 177-181).

Other parameters that may be assessed for this purpose include blood fluidity and blood flow, where an increase in fluidity or flow will generally be indicative of a therapeutically useful effect.

### Methods of Measuring Blood Fluidity

A direct measurement of blood fluidity can be obtained using a Micro Channel Array Flow Analyser (MC-FAN), such as the MC-FAN HR300 available from Arkray, which mimics capillary vessels.

A suitable protocol for use of a MC-FAN is provide in “Determinants of the daily rhythm of blood fluidity”, by Tatsushi Kimura, Tsutomu Inamizu, Kiyokazu Sekikawa, Masayuki Kakehashi and Kiyoshi Onari (Journal of Circadian Rhythms 2009, 7:7).

Briefly microgrooves with width 7 μm, length 30 μm, depth 4.5 μm are formed, for example by photo-fabrication on the surface of a single crystal silicon chip. Suitable chip dimensions may be around 15×15 mm. The microgrooves are then formed into leak-proof microchannels that represent capillaries. This conversion into channels may, for instance, be achieved by tightly covering the channels with a cover such as an optically flat glass plate. Suitable grooves may be transformed into hermetic microchannel by soldering of an optically polished glass plate.

The dimensions of the microchannels are such that the volume of fluid which flows through one flow path is extremely small. Accordingly, it is desirable to replicate the flow channels in order to facilitate measurement of the flow rate. The reference cited above describes the production of a device in which 8736 flow paths of the same size are created. The silicon substrate may then mounted onto the microchannel flow system, MC-FAN (Hitachi Haramachi Electronics Co., Ltd, Ibaragi, Japan), which makes it possible to directly observe the flow of blood cell elements through the microchannel under a microscope connected to an image display unit. Flow can be continuously viewed while the passage time for a given volume of blood is determined automatically.

A suitable value of blood passage may be expressed as a function of the actual whole blood passage time over saline solution passage time of 12 seconds at a pressure of 20 cm H2O, as follows:

$$Blood passage time ( revised value ; sec ) = \frac{Whole blood passage time ( actual value )}{Saline solution passage time} \times 12$$

### Methods of Measuring Blood Flow

Doppler ultrasound flowmetry is a widely used method for assessment of blood flow through intact blood vessels in vivo. Suitable methods using Doppler ultrasound are well known to those skilled in the art, and include those described in “Measurement of blood flow by ultrasound: accuracy and sources of error.” By R. W. Gill (Ultrasound Med Biol. 1985 Jul-Aug; 11(4):625-41).

### DESCRIPTION OF THE DRAWINGS

The invention will now be illustrated, but not limited, by the following examples, and with reference to the accompanying drawings, in which:

FIG. 1: represents examples of dose-response curves of % inhibition of aggregation versus inhibitor solution concentration generated for (a) Compound 1; (b) Compound 5; (c) Compound 9; (d) Compound 18; (e) Compound 23; and (f) Compound 30 as discussed in Example 1, (a) and (b) represent dose-response curves of % inhibition of ADP-mediated aggregation, (c) and (d) represent dose-response curves of % inhibition of collagen-mediated aggregation, (e) and (f) represent dose-response curves of % inhibition of arachidonic acid -mediated aggregation.

FIG. 2 defines a preferred method for making fruit extract enriched in phenolic bioactives.

FIG. 3 defines a preferred method for making low sugar fruit extracts enriched in phenolic bioactives.

FIG. 4: % Change from baseline aggregation in response to different platelet agonists, 3 hours after consumption of tomato extract (TE) or control (C) supplements, as described in Example 4. The platelet agonists used were adenosine diphosphate (ADP) 7.5 μmol/L and 3 μmol/L, and collagen 5 mg/L and 30 mg/L. Significant differences between TE and C supplements are indicated on the graph (P <0.001). N=9 for all measurements.

FIG. 5. Shows average closure times recorded at baseline (0), t=3 hours (3) after supplementation with TE or C and t=5 hours (5) after supplementation with TE or C, as described in Example 5, n=3 for each group. Significant differences between C and TE are indicated on the graph by * (P=0.011).

### EXAMPLE 1

The present invention is based upon research that was conducted to identify bioactive compounds in the fruit extract described in WO 99/55350.

The inventors conducted exhaustive experiments whereby they fractionated tomato extracts to identify compounds within such extracts that were linked to its inhibitory effects on platelet aggregation. The compounds isolated showed a range of antiplatelet activities, and a range of structural types. However the inventors were surprised to find, as discussed below, that a significant proportion of compounds with activity for inhibiting platelet aggregation were phenolic compounds. This led them to realise that such phenolic compounds may be used to treat or prevent the development of conditions according to the invention.

### 1.1. Methods

### 1.1.1 Preparation of a Tomato Extract as Defined by WO 99/55350.

A tomato extract was prepared using commercially available cold-break tomato paste of 28-30 °Brix (i.e. 28-30 % solids, w/w) having a browning index (absorbance of a solution of concentration 12.5 g soluble solids/LL at 420 nm) <0.350 AU as the starting material. The paste was diluted (1:5) with ultrapure water and large particulate matter was removed by centrifugal filtration followed by clarification using a Westfalia MSB-14 Separator (a centrifugal disk clarifier) at room temperature. Smaller particulate matter was then removed by microfiltration at a temperature not exceeding 45° C. to give a clear straw-coloured solution containing no insoluble spin-down solids and capable of passing through a 0.2μ filter without loss of soluble solids. This solution was concentrated by evaporation to a syrup of 65 °Brix, using carefully controlled conditions and a temperature not exceeding 50° C. to limit the progress of non-enzymic browning reactions. A flash pasteurisation step (T=105° C. for 3 seconds) was incorporated at the outset of the evaporation procedure. The final product was characterised by a browning index <0.600 AU, and a microbial total plate count of <1000.

1.1.2 Enrichment of the Tomato Extract with the Active Compounds of Interest and Removal of Inactive Materials

In order to yield a starting material more concentrated in bioactive components, sugars were removed from the product described above as follows.

A 130 L resin column containing FPX66 resin (Rohm and Haas) was prepared and equilibrated in ultrapure water at 4° C. The material described in 1.1.1 was diluted to approximately 8 Brix with ultrapure water, and passed through the resin column at a flow rate of approximately 260 L/minute, maintaining the temperature at 4° C. The column permeate was discarded. Once all the required material had been passed through the column, a water wash of approximately 130 L was passed through and discarded. Thereafter, the compounds which had been retained by the resin were eluted, by passing 130 L of hot water (75° C.) through the columns, followed by 130 L of 80% ethanol, followed by a further 130 L of hot water. All eluted material was retained and combined to give approximately 400 L of approximately 25% ethanolic solution containing the compounds of interest.

The dilute solution containing the compounds of interest was concentrated by reverse osmosis using Trisep ACM5 membranes at temperatures around 30° C. The ethanol/water solvent passed through this membrane, while all compounds dissolved therein remained within the membrane. Once the dilute solution had been concentrated 10-fold, i.e. volume was reduced to 40-50 L, diafiltration commenced, during which ultrapure water was added to the retentate at an equal rate to the permeate removal rate. In this way, the ethanol concentration of the solution was gradually reduced from 25% to <5%.

The ethanolic solution at ˜15-20% solids was then spray-dried using an Anhydro spray-drier to form a fine, golden powder of <6% moisture content. This was the final enriched tomato extract, which was used to isolate antiplatelet components of interest.

### 1.1.3 Isolation and Characterisation of Individual Bioactive Compounds in the Tomato Extract

A stock solution of 50 mg/mL was prepared from the dry powder described in 1.1.2, by dissolving it in ultra-pure HPLC-grade water. Semi-preparative HPLC was carried out using a Luna C18(2) 5μ semi-preparative column, 100×4.6 mm, infecting 100 μL onto the column at a time. Using a fraction collector, the UV-absorbing components contained in the tomato extract were divided into three bulk fractions. Fraction 1 contained largely nucleosides and nucleotides. Fraction 2 contained largely phenolic acid glycosides/esters, and phenolic acids. Fraction 3 contained largely flavonoid glycosides and flavonoids. The three bulk fractions were dried by freeze-drying, and redissolved in water to give solutions of 50 mg/mL. Each fraction in turn was then subjected to further semi-preparative HPLC Using the same column but with different gradients, adapted to the polarity and elution characteristics of each fraction. From each bulk fraction, up to 10 individual or mixed fractions were collected using a fraction collector.

The individual fractions were freeze-dried and redissolved in 1 mL pure water. Each fraction was then examined by analytical HPLC-MS, using a Luna C18(2) 3μ analytical column, 100×4.6 mm, running an acetonitrile/formic acid gradient. Characteristics of each isolated fraction were determined by collection of its UV spectrum via a diode-array detector, and by examination of its characteristic ions generated by electrospray MS in positive ion mode.

Where necessary, final purifications (e.g. to remove minor contaminants) were carried out by further HPLC. Finally purified compounds were freeze-dried and stored frozen. Stock solutions were prepared at 50 mg/mL and diluted into HPLC buffer to produce 6 concentration levels, which were used to calibrate the HPLC method, so that response factors could be calculated for each individual compound. These calibration curves and response factors were then used to quantify the compounds present in the tomato extract. The structural types/identities of the bioactive compounds isolated are shown in Table 2.

### 1.1.4 Methods of Assaying Activity for Inhibiting Platelet Aggregation

The experimental protocol described below was devised to determine the IC50 values of compounds isolated as described in 1.1.3. Crude bioassays to evaluate inhibition of platelet aggregation in vitro were performed on some crude extracts (data not shown) to help select fine fractions/compounds identified by HPLC for functional activity. This approach was considered necessary to avoid the need to assay each and ever compound (the would be thousands) in the fruit extracts.

An IC50 value represents the amount of a compound, in mg, required to inhibit by 50% the platelet aggregation induced under standardised conditions in 1 mL platelet-rich plasma, in comparison with control samples.

The activity of the 32 most active compounds is given in Table 3.

### Phlebotomy and Blood Samples

Blood for in vitro studies was collected from drug-free, healthy human volunteers, both male and female, aged 18-60. years, with normal platelet function. Subjects declared that they had not consumed drugs or supplements known to affect platelet function for a minimum of 10 days before providing a blood sample. Blood was collected after single venepuncture to an antecubital vein through siliconized needles into plastic citrated blood collection tubes (Sarstedt Monovettes, final concentration sodium citrate, 13 mmol/L). All blood was maintained at 37° C. from the time of blood sampling.

### Preparation of Platelet-Rich Plasma

Platelet-rich plasma (PRP) was obtained by centrifugation of citrated blood for 15 minutes at 200×g, and was adjusted with platelet-poor plasma to a standard platelet number of 320±20×10⁹/L prior to use. PRP was used for platelet function measurements within two hours.

### Platelet Agonists

The following agonists were used for platelet function measurements. Adenosine diphosphate (ADP), final concentration 10 μmol/L; collagen, final concentration 5 mg/L; arachidonic acid, final concentration 500 U/L (all from Helena Biosciences, Sunderland, UK); thrombin receptor-activating peptide (TRAP), final concentration 25 nmol/L (Sigma-Aldrich, Poole, UK). Agonists were prepared from stock solutions immediately before use, diluting into warmed physiological saline (0.9% NaCl).

### Preparation of Platelet Inhibitor Solutions

Individual platelet inhibitors were prepared at a concentration of between 500 g/L and 100 g/L in either physiological saline, ultra-pure methanol or ultra-pure DMSO (Sigma-Aldrich, Poole, UK) and stored frozen until required. Stock solutions were then diluted with physiological saline immediately prior to use.

Incubation of Platelet Inhibitors with PRP

450 μL PRP was incubated with 50 μL diluted inhibitor solution at 37° C. for 10 minutes, in low-retention epindorrfs. Inhibitor solutions were diluted such that the final concentration of methanol or DMSO In the PRP sample never exceeded 2%. Suitable control samples, containing 50 μL physiological saline matched for methanol or DMSO content as appropriate, were incubated simultaneously. For each inhibitor compound, 5 incubation concentrations were used; final concentrations of 0.05 mg/mL, 0.10 mg/mL, 1.00 mg/mL, 5.00 mg/mL and 10 mg/mL were used as standard.

### Measurement of Platelet Aggregation and Inhibition of Aggregation

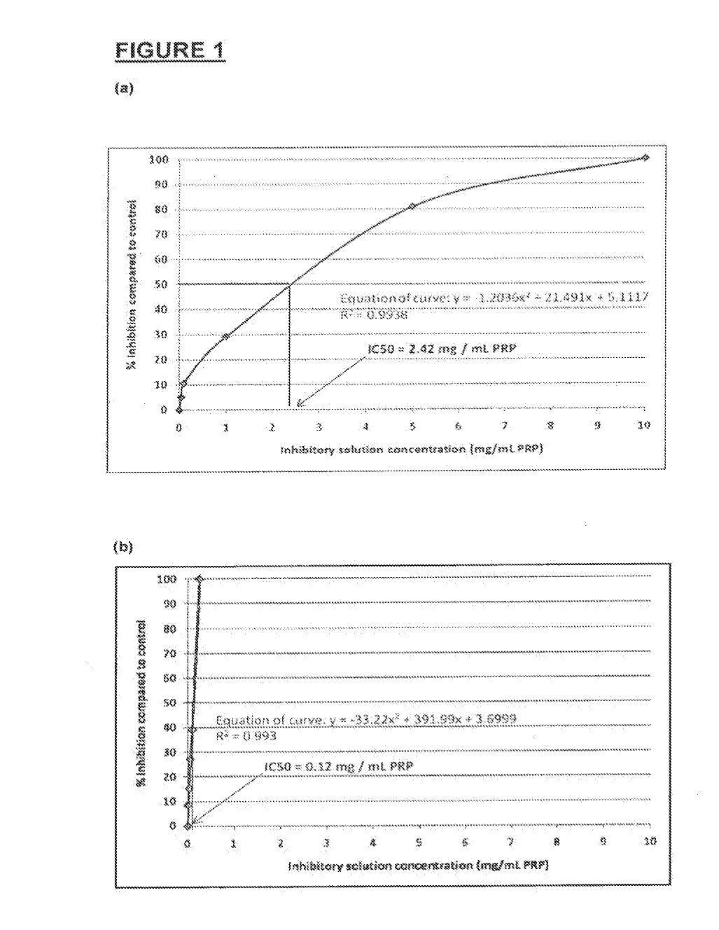

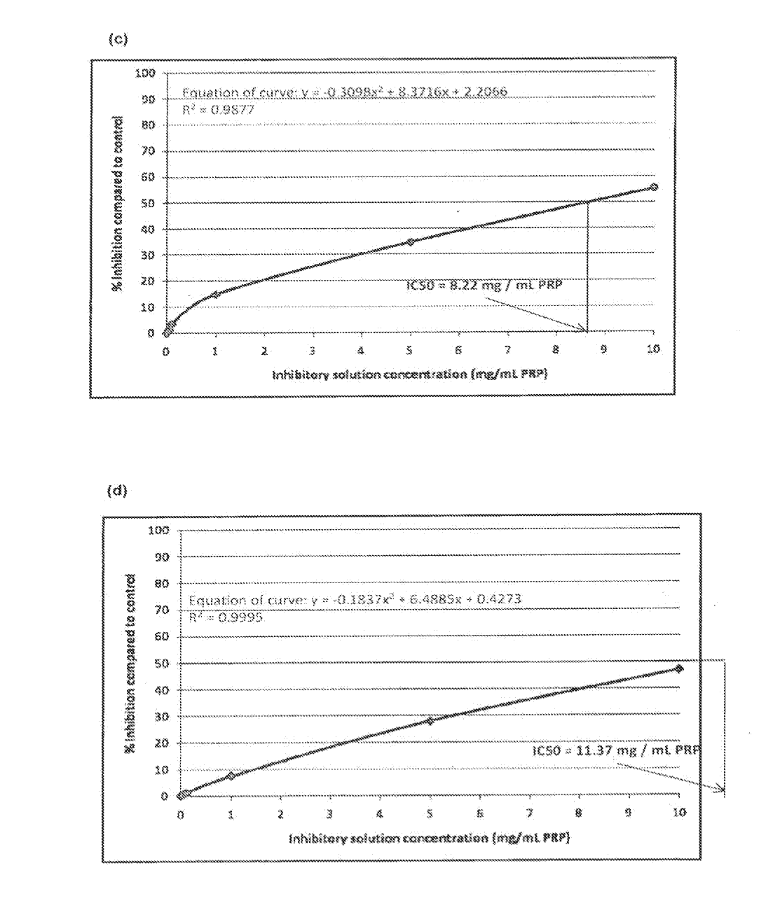

After incubation with platelet inhibitors, PRP samples were transferred to glass cuvettes and the extent of aggregation induced by either ADP, collagen, TRAP or arachidonic acid was monitored over 10 minutes on a platelet aggregometer (PACKS 4, Helena Biosciences, Sunderland, UK). A control sample was run with each sample set. From the aggregation curves generated, the area under the curve was calculated for each PRP sample, and the inhibition of aggregation achieved at each inhibitor concentration was calculated by comparing the area under the curve for these PRP samples with that of the control sample. The inhibition of aggregation was expressed as % inhibition, compared to control, and from the 6 data points obtained per inhibitor compound, a dose-response curve was constructed. This curve was then used to predict the IC50 value for that inhibitor compound, as shown in 1.2, Results, and FIG. 1.

For each blood sample obtained, 6-point dose-response curves for 2 different inhibitory compounds could be generated. These experiments were repeated such that for each inhibitory compound, at feast 3 (most often 7-10) different IC50 values were obtained on different days, using blood from different subjects (this applies to each agonist of interest). An average of the different IC50s was then taken and these values are quoted in 1.2, Results, Table 3.

### 1.2 Results

The physiochemical properties of the 32 compounds, found to have most antiplatelet activity (see below) are summarised in Table 2.

TABLE 2
Physiochemical Properties of Bioactive Compounds identified in Fruit Extracts

| | Compound | | | | Mass/characteristic ions |
|---|---|---|---|---|---|
| Group | ID | Bioactive Compound | RT (s) | λ max | (POS mode) |
| Nucleosides | 1 | Cytidine | 1.24 | 275 | 487, 244 |
| | 2 | Adenosine | 3.17 | 260 | 268, 136 |
| | 3 | Uridine | 2.59 | 270 | 267, 113 |
| | 4 | Guanosine | 3.9 | 260 (278sh) | 284, 152 |
| Nucleotides | 5 | Adenosine 3′- | 1.6 | 260 | 348, 136 |
| | | monophospate | | | |
| | 6 | Adenosine 5′- | 1.78 | 260 | 348, 136 |
| | | monophospate | | | |
| Phenolic acid | 7 | Mixed phenolic acid | 8.0-9.0 | | Mixture |
| glycosides | | glycosides | | | |
| | 8 | p-Coumaric acid hexose/ | 9.02 | 300 | 469, 147, 119 |
| | | quinic acid derivative | | | |
| | 9 | Caffeic acid glucoside | 9.39 | 290 | 319, 163 |
| | 10 | Ferulic acid hexose | 9.67 | 295, 315 | 265, 177 |
| | 11 | p-Coumaric acid hexose/ | 10.62 | 265 | 467, 449, 287; 450, 163 |
| | | dihydrokaempferol | | | |
| | | hexose mixture | | | |
| | 12 | p-Coumaric acid/ | 11 | 285 | 367, 344, 163, 147 |
| | | caffeic acid conjugate, | | | |
| | | glycosylated | | | |
| | 13 | Ferulic acid glycoside | 11 | 285, 315 sh | 379, 196, 177 |
| | 14 | Chlorogenic acid | 12.77 | 325, 300sh | 163, 377 |
| Phenolic ester | 15 | p-Coumaric acid | 11.55 | 275 | 396, 196, 163 |
| derivatives | | derivative | | | |
| | 16 | Caffeoyl-quinic acid | 14.96 | 310 | |
| | | dimer #1 | | | |
| | 17 | Caffeoyl-quinic acid | 26.63 | | 573, 814, 163 |
| | | dimer #2 | | | |
| Phenolic | 18 | Caffeic acid | 13.39 | 325, 295 sh | 163 |
| acids | 19 | p-coumaric acid | 18.19 | 235, 310 | 165, 147, 119 |
| | 20 | Benzoic acid | 22.36 | | |
| | 21 | Ferulic acid | 22.61 | | 177 |
| | 22 | Cinnamic acid | 30.27 | 273 | 621, 599.5, 131.1 |
| Flavonoid | 23 | Quercetin-3-O- | 23.57 | 275 | 400, 303 |
| glycosides | | glycoside | | | |
| | 24 | Kaempferol glycoside | 24.7 | | 592, 535 |
| | 25 | Quercetin-3-O- | 25.44 | | 765, 453, 303 |
| | | trisaccharides | | | |
| | 26 | Naringin | 25.88 | 285, 330 | 621, 563 |
| | 27 | Rutin | 27.2 | 260, 350 | 633, 303 |
| Flavonoid | 28 | Flavonoid conjugate | 24.33 | | 258 |
| ester | 29 | Trace flavonoids + | 27.5-30.0 | | Mixture |
| derivatives | | glycosides | | | |
| Flavonoids | 30 | Quercetin | 36.5 | 255, 370 | 629, 303, 273 |
| | 31 | Kaempferol | 44.58 | 260, 370 | 287 |
| | 32 | Naringenin | 35.1 | | |

Table 3 provides IC50 data (for inhibiting platelet aggregation) for the 32 most active compounds identified in the tomato extract. Activity was assayed as described in Method 1.1.4. FIG. 1 provides examples of dose-response curves of % inhibition of platelet aggregation versus inhibitor solution concentration generated for (a) a nucleoside (cytidine); (b) a nucleotide (adenosine 3′ monophosphate; (c) a phenolic acid glycoside (Caffeic acid glucoside); (d) a phenolic acid (Caffeic acid); (e) a flavonoid glycoside (Quercetin-3-O-glycoside); and (f) a flavonoid (Quercetin).

TABLE 3
Antiplatelet Activity of Compounds identified in Fruit Extracts

| | Compound | | | IC50 | IC50 | |
|---|---|---|---|---|---|---|
| Group | ID | Bioactive Compound | IC50 ADP | Collagen | TRAP | IC50 AA |
| Nucleosides | 1 | Cytidine | 2.42 | 10.66 | 39.03 | 39.03 |
| | 2 | Adenosine | 0.4 | 0.82 | >50 | — |
| | 3 | Uridine | 6.51 | 15.99 | >50 | — |
| | 4 | Guanosine | 0.25 | 0.53 | 26.07 | 0.91 |
| Nucleotides | 5 | Adenosine 3′- | 0.12 | 0.28 | 24.51 | 2.41 |
| | | monophospate | | | | |
| | 6 | Adenosine 5′- | 0.12 | 0.28 | 24.51 | 2.41 |
| | | monophospate | | | | |
| Phenolic acid | 7 | Mixed phenolic acid | N/A | N/A | N/A | N/A |
| glycosides | | glycosides | | | | |
| | 8 | p-Coumaric acid hexose/ | 10.25 | 9.88 | 1.61 | 0.19 |
| | | quinic acid derivative | | | | |
| | 9 | Caffeic acid glucoside | 10.16 | 8.22 | 0.8 | 0.23 |
| | 10 | Ferulic acid hexose | 12.61 | 14.16 | 0.52 | 0.46 |
| | 11 | p-Coumaric acid hexose/ | 11.1 | 14 | 0.56 | 0.31 |
| | | dihydrokaempferol | | | | |
| | | hexose mixture | | | | |
| | 12 | p-Coumaric acid/ | 12.61 | 13.18 | 0.25 | 0.2 |
| | | caffeic acid conjugate, | | | | |
| | | glycosylated | | | | |
| | 13 | Ferulic acid glycoside | 13.11 | 14.56 | 0.37 | 0.41 |
| | 14 | Chlorogenic acid | 10.08 | 10.11 | 1.1 | 0.77 |
| Phenolic ester | 15 | p-Coumaric acid | 14.65 | 15.18 | 0.35 | 0.26 |
| derivatives | | derivative | | | | |
| | 16 | Caffeoyl-quinic acid | 31.55 | 35 | 11.12 | 0.3 |
| | | dimer #1 | | | | |
| | 17 | Caffeoyl-quinic acid | 32.96 | 33.07 | 12.16 | 0.2 |
| | | dimer #2 | | | | |
| Phenolic | 18 | Caffeic acid | 18.98 | 11.37 | 8.03 | 7.33 |
| acids | 19 | p-coumaric acid | 13.22 | 14.62 | 12.82 | 10.18 |
| | 20 | Benzoic acid | 25.11 | 17.74 | 18.19 | 15.45 |
| | 21 | Ferulic acid | 18.67 | 13.9 | 14.65 | 9.94 |
| | 22 | Cinnamic acid | 22.14 | 24.6 | 12.92 | 0.22 |
| Flavonoid | 23 | Quercetin-3-O- | 25.18 | 28.43 | 12.06 | 0.19 |
| glycosides | | glycoside | | | | |
| | 24 | Kaempferol glycoside | >50 | >50 | N/A | N/A |
| | 25 | Quercetin-3-O- | >50 | >50 | 18.61 | 0.46 |
| | 26 | Naringin | 28.1 | 29.55 | 9.13 | 0.31 |
| | 27 | Rutin | 35.21 | 32.18 | 8.96 | 0.41 |
| Flavonoid | 28 | Flavonoid conjugate | 27.68 | 27.22 | 13.67 | 0.23 |
| ester | 29 | Trace flavonoids + | N/A | N/A | N/A | N/A |
| derivatives | | glycosides | | | | |
| Flavonoids | 30 | Quercetin | >50 | >50 | 19.66 | 3.66 |
| | 31 | Kaempferol | >50 | >50 | 26.18 | 5.18 |
| | 32 | Naringenin | >50 | >50 | 33.21 | 10.41 |

Having established that phenolic and flavonoid compounds derivable from tomato extracts had antiplatelet activity, the inventors went on to obtain and test bioactives from other sources (whether other plants or chemical suppliers). Table 4 presents data for such compounds and illustrates that the classes of bioactive compounds first identified in tomato extracts also have activity if derived from a different source. The compounds identified in Table 4 represent preferred bioactive compounds that may be used according to the invention.

TABLE 4
Antiplatelet compounds of phenolic/flavonoid structure
derived from sources other than tomatoes

| | IC50 | IC50 | IC50 | IC50 |
|---|---|---|---|---|
| Compound | ADP | Collagen | TRAP | AA |
| Protocatechuic acid | 30.16 | 23.66 | 30.77 | 25.61 |
| Gallic acid | 20.21 | 14.38 | 18.95 | 14.44 |
| Syringic acid | 37.18 | 30.75 | 28.26 | 22.47 |
| Cinnamic acid | 26.11 | 8.76 | 18.18 | 16.11 |
| o-Coumaric acid | 32.19 | 28.91 | 17.38 | 9.48 |
| Sinapic acid | 26.11 | 24.89 | 30.36 | 24.75 |
| Myricetin | 22.14 | 24.6 | 12.92 | 0.22 |
| Quercitrin | >50 | >50 | 15.98 | 12 |
| Luteolin | >50 | >50 | 26.18 | 5.18 |
| Inosine | 3.5 | 9.3 | >50 | — |
| Guanosine | 0.1 | 0.31 | 20.11 | 0.36 |
| 5′-monophosphate | | | | |

### 1.3 Conclusions

The inventors tested many compounds found within tomato extracts and established that the 32 compounds identified above had efficacy for preventing platelet aggregation.

In particular they were surprised to find that a significant proportion of the bioactive compounds (compounds 7-22 above) were phenolic compounds (and ester and glycoside derivatives thereof). This lead them to realise that a new class of compounds (the phenolics) exists which has an inhibitory effect on platelet aggregation and may be used according to the invention.

Of the phenolic acid-derived compounds identified, the most anti-aggregatory overall were the glycosylated forms of p-coumaric and caffeic acids. These glycosylated compounds showed markedly higher anti-aggregatory potential in response to all agonists tested, compared to the non-glycosylated free acids. This is the first time such a structure-function relationship has been reported. Accordingly glycosylated phenolic compounds represent most preferred bioactive molecules that may be contained with the compositions or extracts according to the invention and which should be maintained/enriched in extracts prepared according to the method the invention.

Other groups of compounds were identified as having bioactivity and may be included in preferred compositions according to the invention. These include flavonoids and ester and glycoside derivatives thereof (compounds 23-32 above); and nucleotides/nucleosides (compounds 1-6) above.

The inventors found that flavonoid glycosides or other conjugated derivatives of quercetin and naringenin were markedly more anti-aggregatory than the flavonoid aglycones. This was particularly noticeable in response to TRAP and arachidonic acid agonists, but also applied to ADP and collagen agonists. While a (very limited) amount of structure-function studies have been reported in the literature for the free flavonoid aglycones, the authors are unaware of any studies comparing aglycones and conjugated molecules. Accordingly glycosylated flavonoid compounds also represent most preferred bioactive molecules that may be contained with the compositions and extracts according to the invention and which should be maintained/enriched in extracts prepared according to the method of the first aspect of the invention.

### EXAMPLE 2

Experiments were conducted to investigate the activity of the three “bioactive” fractions identified during the work carried out for Example 1 (see 1.1.3).

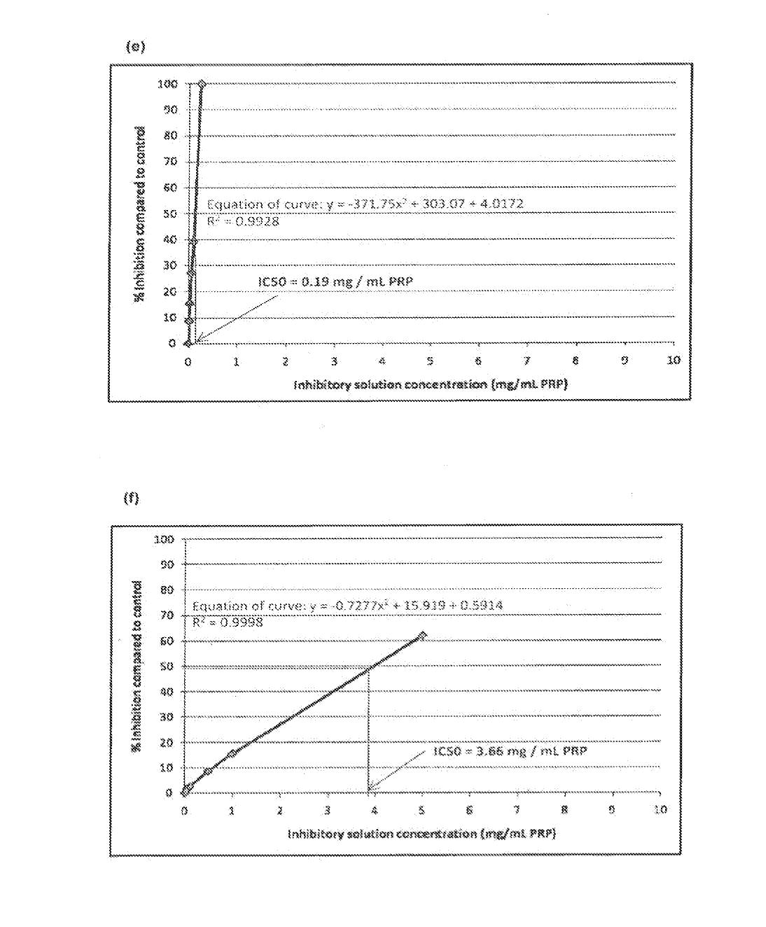

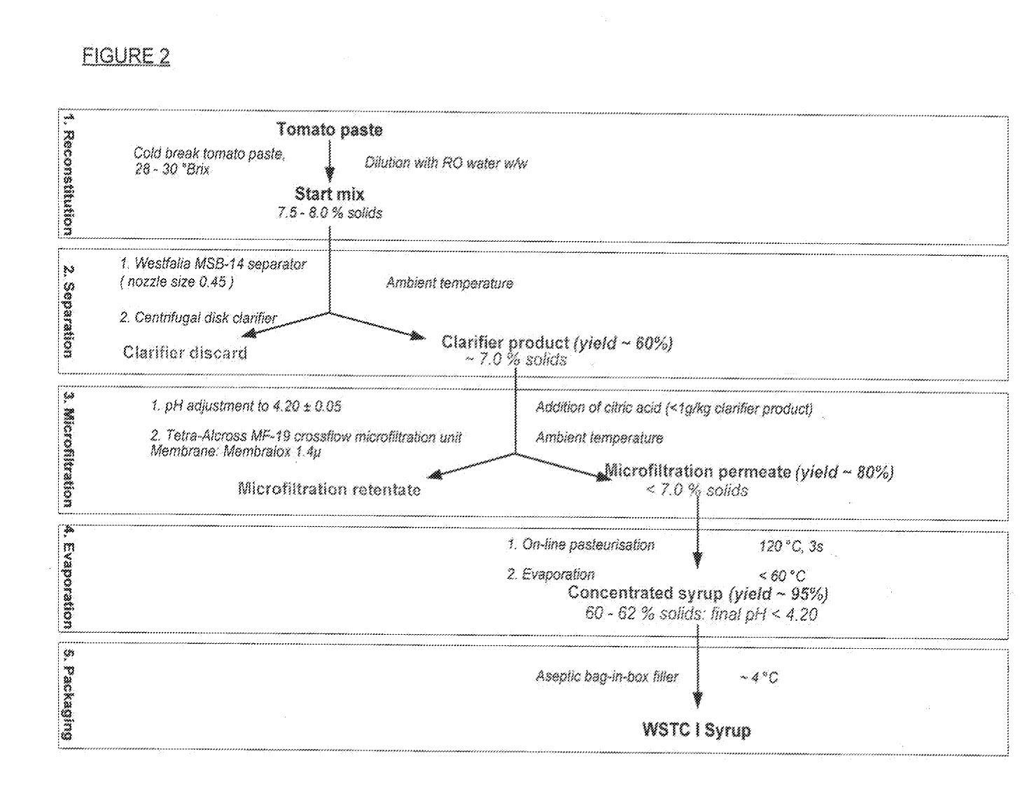

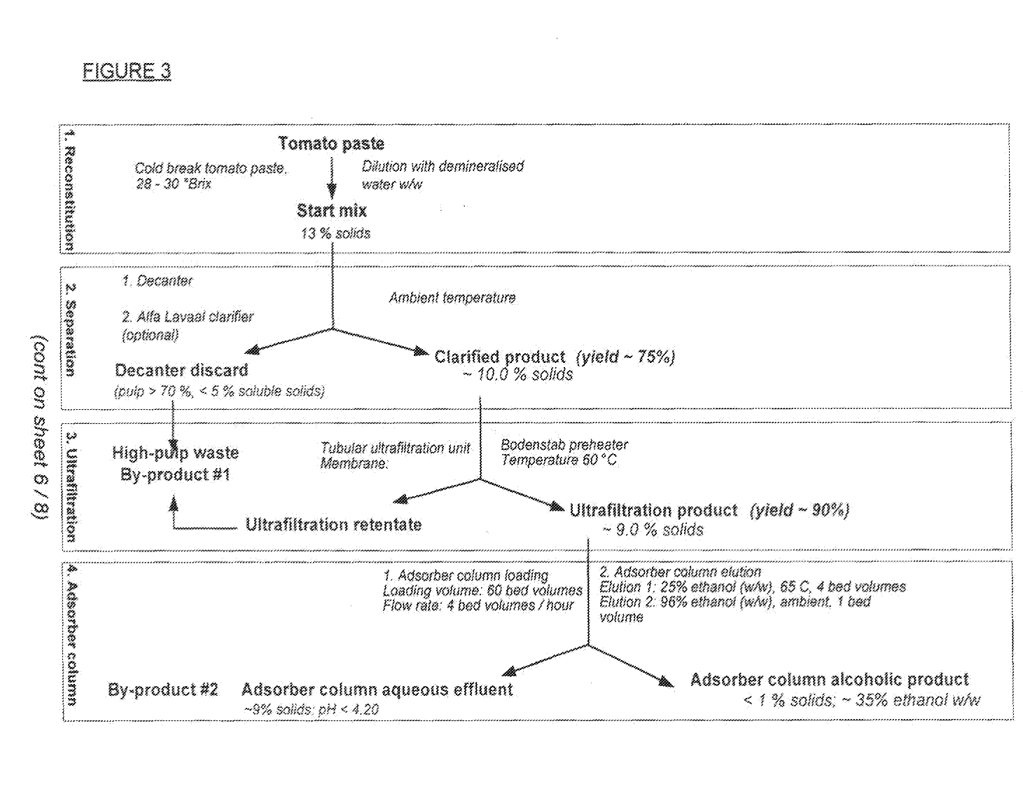

Fraction 2 referred to in Example 1 was separately tested to determine its antiplatelet profile. This represents a preferred mixture enriched specifically in phenolic acids and derivatives. Table 5 shows the composition of Fraction 2 when isolated from fruit extracts prepared according to the methods described in FIGS. 2 and 3, while Table 6 shows the antiplatelet activity of the mixture.

TABLE 5
Phenolic acids contained in Fraction 2, derived from tomatoes.

| | | | Fraction 2 from an | | | Fraction 2 from an extract | | |
|---|---|---|---|---|---|---|---|---|
| | | | extract prepared | | | prepared according to | | |
| | | | according to procedure | | | procedure described in FIG. | | |
| | | | described in FIG. 2 | | | 3 (low sugar extract) | | |
| | | | lower | upper | | lower | upper | |
| | | | range | range | average | range | range | average |
| Group | ID# | Compound | mg/g | mg/g | mg/g | mg/g | mg/g | mg/g |
| Phenolic | 7 | Mixed phenolic acid | 0.352 | 0.956 | 0.796 | 20.982 | 145.537 | 121.281 |
| acid | | glycosides | | | | | | |
| glycosides | 8 | p-Coumaric acid hexose/ | 0.050 | 0.456 | 0.380 | 9.418 | 11.867 | 9.889 |
| | | quinic acid derivative | | | | | | |
| | 9 | Caffeic acid glucoside | 0.069 | 0.477 | 0.398 | 3.736 | 13.402 | 11.168 |
| | 10 | Ferulic acid hexose | 0.028 | 0.048 | 0.040 | 0.706 | 1.340 | 1.117 |
| | 11 | p-Coumaric acid hexose/ | 0.277 | 0.997 | 0.831 | 26.121 | 40.288 | 33.573 |
| | | dihydrokaempferol hexose | | | | | | |
| | | mixture | | | | | | |
| | 12 | p-Coumaric acid/ caffeic | 0.170 | 1.419 | 1.182 | 90.872 | 131.722 | 109.768 |
| | | acid conjugate, | | | | | | |
| | | glycosylated | | | | | | |
| | 13 | Ferulic acid glycoside | 0.155 | 1.199 | 0.999 | 85.333 | 199.679 | 166.399 |
| | 14 | Chlorogenic acid | 0.131 | 0.953 | 0.794 | 18.274 | 43.366 | 36.138 |
| Phenolic | 15 | p-Coumaric acid | 0.105 | 0.332 | 0.277 | 8.620 | 16.584 | 13.820 |
| ester | | derivative | | | | | | |
| derivatives | 16 | Caffeoyl-quinic acid | 0.066 | 0.701 | 0.584 | 13.850 | 85.176 | 70.980 |
| | | dimer #1 | | | | | | |
| | 17 | Caffeoyl-quinic acid | 0.142 | 0.701 | 0.584 | 12.672 | 22.731 | 18.943 |
| | | dimer #2 | | | | | | |
| Phenolic | 18 | Caffeic acid | 0.058 | 0.873 | 0.727 | 5.842 | 9.042 | 7.535 |
| acids | 19 | p-coumaric acid | 0.046 | 0.488 | 0.407 | 11.403 | 27.568 | 22.974 |
| | 20 | Benzoic acid | 0.006 | 0.077 | 0.064 | 0.959 | 1.554 | 1.295 |
| | 21 | Ferulic acid | 0.016 | 0.140 | 0.117 | 0.584 | 1.113 | 0.927 |
| | 22 | Cinnamic acid | 0.028 | 0.084 | 0.070 | 1.966 | 6.896 | 5.747 |

TABLE 6
IC50 of the Fraction 2 mixture of phenolic
acids and derivatives isolated from tomato.

| | | IC50 | IC50 | IC50 | IC50 |
|---|---|---|---|---|---|
| | Description | ADP | Collagen | TRAP | AA |
| Fraction 2 | Extract enriched in | 0.4 | 0.21 | 0.2 | 0.5 |
| | Phenolic acids and | | | | |
| | derivatives theeof | | | | |

In addition, Fractions 2 and Fraction 3 (flavonoid fraction) referred to in Example 1 were combined to provide a composition enriched in phenolic acid derivatives and flavonoids. This mixture was examined for antiplatelet activity as described above. Its composition is shown in Table 7, and the antiplatelet activities are shown in Table 8.

TABLE 7
Composition of Mixture 1 obtained by mixing Fraction 2 and
Fraction 3 derived from tomatoes.

| | | | Combined | | | Combined Fractions 2 | | |
|---|---|---|---|---|---|---|---|---|
| | | | Fractions | | | and Combined | | |
| | | | 2 and 3 from | | | 3 from an extract prepared | | |
| | | | an extract prepared | | | according to procedure | | |
| | | | according to procedure | | | described in FIG. 3 (low | | |
| | | | described in FIG. 2 | | | sugar) | | |
| | | | lower | upper | | lower | upper | |
| | | | range | range | average | range | range | average |
| Group | ID# | Compound | mg/g | mg/g | mg/g | mg/g | mg/g | mg/g |
| Phenolic acid | 7 | Mixed phenolic acid | 0.352 | 0.956 | 0.796 | 20.982 | 145.537 | 121.281 |
| glycosides | | glycosides | | | | | | |
| | 8 | p-Coumaric acid hexose/ | 0.050 | 0.456 | 0.380 | 9.418 | 11.867 | 9.889 |
| | | quinic acid derivative | | | | | | |
| | 9 | Caffeic acid glucoside | 0.069 | 0.477 | 0.398 | 3.736 | 13.402 | 11.168 |
| | 10 | Ferulic acid hexose | 0.028 | 0.048 | 0.040 | 0.706 | 1.340 | 1.117 |
| | 11 | p-Coumaric acid hexose/ | 0.277 | 0.997 | 0.831 | 26.121 | 40.288 | 33.573 |
| | | dihydrokaempferol | | | | | | |
| | | hexose mixture | | | | | | |
| | 12 | p-Coumaric acid/ | 0.170 | 0.419 | 1.182 | 90.872 | 131.722 | 109.768 |
| | | caffeic acid conjugate, | | | | | | |
| | | glycosylated | | | | | | |
| | 13 | Ferulic acid glycoside | 0.155 | 1.199 | 0.999 | 85.333 | 199.679 | 166.399 |
| | 14 | Chlorogenic acid | 0.131 | 0.953 | 0.794 | 18.274 | 43.366 | 36.138 |
| Phenolic ester | 15 | p-Coumaric acid | 0.105 | 0.332 | 0.277 | 8.620 | 16.584 | 13.820 |
| derivatives | | derivative | | | | | | |
| | 16 | Caffeoyl-quinic acid | 0.066 | 0.701 | 0.584 | 13.850 | 85.176 | 70.980 |
| | | dimer #1 | | | | | | |
| | 17 | Caffeoyl-quinic acid | 0.142 | 0.701 | 0.584 | 12.672 | 22.731 | 18.943 |
| | | dimer #2 | | | | | | |
| Phenolic acids | 18 | Caffeic acid | 0.058 | 0.873 | 0.727 | 5.842 | 9.042 | 7.535 |
| | 19 | p-coumaric acid | 0.046 | 0.488 | 0.407 | 11.403 | 27.568 | 22.974 |
| | 20 | Benzoic acid | 0.006 | 0.077 | 0.064 | 0.959 | 1.554 | 1.295 |
| | 21 | Ferulic acid | 0.016 | 0.140 | 0.117 | 0.584 | 1.113 | 0.927 |
| | 22 | Cinnamic acid | 0.028 | 0.084 | 0.070 | 1.966 | 6.896 | 5.747 |
| Flavonoid | 23 | Quercetin-3-O- | 0.050 | 0.324 | 0.270 | 8.463 | 13.257 | 11.048 |
| glycosides | | glycoside | | | | | | |
| | 24 | Kaempferol glycoside | 0.008 | 0.049 | 0.041 | 1.269 | 5.277 | 4.398 |
| | 25 | Quercetin-3-O- | 0.157 | 0.610 | 0.508 | 14.679 | 24.799 | 20.666 |
| | | trisaccharides | | | | | | |
| | 26 | Naringin | 0.739 | 2.103 | 1.753 | 38.016 | 61.709 | 51.424 |
| | 27 | Rutin | 0.583 | 2.804 | 2.337 | 50.688 | 106.147 | 88.456 |
| Flavonoid | 28 | Flavonoid conjugate | 0.004 | 0.032 | 0.027 | 0.846 | 1.733 | 1.444 |
| ester | 29 | Trace flavonoids + | 1.253 | 3.900 | 3.250 | 90.660 | 319.469 | 266.224 |
| derivatives | | glycosides | | | | | | |
| Flavonoids | 30 | Quercetin | 0.014 | 0.130 | 0.108 | 3.787 | 20.578 | 17.149 |
| | 31 | Kaempferol | 0.039 | 0.180 | 0.150 | 3.749 | 8.230 | 6.858 |
| | 32 | Naringenin | trace | 1.540 | trace | trace | 25.600 | trace |

TABLE 8
IC50 of the Mixture 1 combination comprising tomato-derived
phenolic acid derivatives and flavonoid derivatives.

| | | IC50 | IC50 | IC50 | IC50 |
|---|---|---|---|---|---|
| Mixture | Description | ADP | Collagen | TRAP | AA |
| Mixture 1 | Phenolic acids and | 0.25 | 0.24 | 0.15 | 0.7 |
| | derivatives combined | | | | |
| | with flavonoids and | | | | |
| | derivatives | | | | |

A further composition was created by combining Fractions 1, 2 and 3 from 1.1.3 above,. Such a composition represents a preferred mixture containing phenolic acids, flavonoids and nucleosides. Its composition is shown in Table 9 and its antiplatelet activity was determined as described above and results are shown in Table 10.

TABLE 9
Composition of Mixture 2, obtained by mixing Fractions 1, 2 and 3 derived from tomatoes

| | | | Combined | | | Combined | | |
|---|---|---|---|---|---|---|---|---|
| | | | Fractions 1, 2 and | | | Fractions 1, 2 and | | |
| | | | 3 from an extract | | | 3 from an extract prepared | | |
| | | | prepared according to | | | according to procedure | | |
| | | | procedure described in | | | described in FIG. 3 (low | | |
| | | | FIG. 2 | | | sugar) | | |
| | | | lower | upper | | lower | upper | |
| | | | range | range | average | range | range | average |
| Group | ID# | Compound | mg/g | mg/g | mg/g | mg/g | mg/g | mg/g |
| Nucleosides | 1 | Cytidine | 0.487 | 2.051 | 1.709 | 21.971 | 36.911 | 30.759 |
| | 2 | Adenosine | 0.382 | 2.440 | 2.033 | 1.800 | 2.927 | 2.439 |
| | 3 | Uridine | 0.414 | 2.089 | 1.741 | 21.917 | 31.340 | 26.117 |
| | 4 | Guanosine | 0.400 | 1.759 | 1.466 | 6.970 | 19.354 | 16.128 |
| Nucleotides | 5 | Adenosine 3′- | 1.312 | 11.491 | 9.576 | 6.421 | 16.087 | 13.406 |
| | | monophospate | | | | | | |
| | 6 | Adenosine 5′- | | | | | | |
| | | monophospate | | | | | | |
| Phenolic acid | 7 | Mixed phenolic acid | 0.352 | 0.956 | 0.796 | 20.982 | 145.537 | 121.281 |
| glycosides | | glycosides | | | | | | |
| | 8 | p-Coumaric acid hexose/ | 0.050 | 0.456 | 0.380 | 9.418 | 11.867 | 9.889 |
| | | quinic acid derivative | | | | | | |
| | 9 | Caffeic acid glucoside | 0.069 | 0.477 | 0.398 | 3.736 | 13.402 | 11.168 |
| | 10 | Ferulic acid hexose | 0.028 | 0.048 | 0.040 | 0.706 | 1.340 | 1.117 |
| | 11 | p-Coumaric acid hexose/ | 0.277 | 0.997 | 0.831 | 26.121 | 40.288 | 33.573 |
| | | dihydrokaempferol | | | | | | |
| | | hexose mixture | | | | | | |
| | 12 | p-Coumaric acid/ | 0.170 | 1.419 | 1.182 | 90.872 | 131.722 | 109.768 |
| | | caffeic acid conjugate, | | | | | | |
| | | glycosylated | | | | | | |
| | 13 | Ferulic acid glycoside | 0.155 | 1.199 | 0.999 | 85.333 | 199.679 | 166.399 |
| | 14 | Chlorogenic acid | 0.131 | 0.953 | 0.794 | 18.274 | 43.366 | 36.138 |
| Phenolic ester | 15 | p-Coumaric acid | 0.105 | 0.332 | 0.277 | 8.620 | 16.584 | 13.820 |
| derivatives | | derivative | | | | | | |
| | 16 | Caffeoyl-quinic acid | 0.066 | 0.701 | 0.584 | 13.850 | 85.176 | 70.980 |
| | | dimer #1 | | | | | | |
| | 17 | Caffeoyl-quinic acid | 0.142 | 0.701 | 0.584 | 12.672 | 22.731 | 18.943 |
| | | dimer #2 | | | | | | |
| Phenolic acids | 18 | Caffeic acid | 0.058 | 0.873 | 0.727 | 5.842 | 9.042 | 7.535 |
| | 19 | p-coumaric acid | 0.046 | 0.488 | 0.407 | 11.403 | 27.568 | 22.974 |
| | 20 | Benzoic acid | 0.006 | 0.077 | 0.064 | 0.959 | 1.554 | 1.295 |
| | 21 | Ferulic acid | 0.016 | 0.140 | 0.117 | 0.584 | 1.113 | 0.927 |
| | 22 | Cinnamic acid | 0.028 | 0.084 | 0.070 | 1.966 | 6.896 | 5.747 |
| Flavonoid | 23 | Quercetin-3-O- | 0.050 | 0.324 | 0.270 | 8.463 | 13.257 | 11.048 |
| glycosides | | glycoside | | | | | | |
| | 24 | Kaempferol glycoside | 0.008 | 0.049 | 0.041 | 1.269 | 5.277 | 4.398 |
| | 25 | Quercetin-3-O- | 0.157 | 0.610 | 0.508 | 14.679 | 24.799 | 20.666 |
| | | trisaccharides | | | | | | |
| | 26 | Naringin | 0.739 | 2.103 | 1.753 | 38.016 | 61.709 | 51.424 |
| | 27 | Rutin | 0.583 | 2.804 | 2.337 | 50.688 | 106.147 | 88.456 |
| Flavonoid | 28 | Flavonoid conjugate | 0.004 | 0.032 | 0.027 | 0.846 | 1.733 | 1.444 |
| ester | 29 | Trace flavonoids + | 1.253 | 3.900 | 3.250 | 90.660 | 319.469 | 266.224 |
| derivatives | | glycosides | | | | | | |
| Flavonoids | 30 | Quercetin | 0.014 | 0.130 | 0.108 | 3.787 | 20.578 | 17.149 |
| | 31 | Kaempferol | 0.039 | 0.180 | 0.150 | 3.749 | 8.230 | 6.858 |
| | 32 | Naringenin | trace | 1.540 | trace | trace | 25.600 | trace |

TABLE 10
IC50 of the Mixture 2 combination comprising tomato-derived phenolic
acid and flavonoid derivatives, and nucleosides/nucleotides.

| | | IC50 | IC50 | IC50 | IC50 |
|---|---|---|---|---|---|
| Mixture | Description | ADP | Collagen | TRAP | AA |
| F1 + | Combination of phenolic | <0.05 | <0.05 | <0.5 | <0.7 |
| F2 + F3 | acids and derivatives, | | | | |
| | flavonoids and | | | | |
| | derivatives, and | | | | |
| | nucleosides/nucleotides | | | | |

It should be noted that although tomato has been used as the source of Fractions 1, 2 and 3 in this instance, other fruit sources could be used. Using an identical methodology, this would result in Fractions 1, 2 and 3 with different phenolic acid derivative and flavonoid derivative compositions that would be specific to the plant source.

From these experiments, the authors concluded that [Fraction 1+Fraction 2] had a wider range of antiplatelet activities than Fraction 2 alone, and that [Fraction 1+Fraction 2+Fraction 3] had a wider range of activities than Mixture 1. Thus the combination of the different compound types resulted in a more broadly active antiplatelet mixture.

### EXAMPLE 3

In the following Example, an experiment is described in which the anti-platelet efficacy of a composition, enriched in phenolic bioactives according to the invention, was assessed.

### 3.1 Study Protocol

### 3.1.1 Study Objectives and Short Outline

This study quantified the ex vivo anti-platelet effect of consuming a treatment drink containing 3 g of tomato extract syrup (prepared according to the methods described FIG. 2), compared to a control supplement, in healthy subjects.

### 3.1.2 Study Design

A single-blinded study design was followed. Fasted subjects were cannulated and a baseline sample was taken between 07:00 and 08:00. Directly after collection of the baseline sample, subjects consumed either a treatment (TE) or a control supplement. Further blood samples were then withdrawn from the cannula at time t=3 hours. Subjects were offered small volumes (25 mL) of water between sampling time points to avoid dehydration.

### 3.1.3 Subjects

9 healthy adults of both sexes were recruited into the study. Subjects were aged 40-65 years, with no medical history of serious disease or hemostatic disorders. Suitability for inclusion onto the study was assessed by diet and lifestyle questionnaires and medical screening, during which a full blood count was obtained. Individuals with low hematology counts were not included in the study. Any subjects habitually consuming dietary supplements (e.g. fish oils, evening primrose oil) suspended these supplements for a minimum period of one month before participating in the study. Subjects were instructed to abstain from consuming drugs known to affect platelet function for a 10-day period prior to participation. Written informed consent was obtained from all subjects, and the study was approved by Grampian Research Ethics Committee.

### 3.1.4 Phlebotomy

Subjects recruited into the study were cannulated using a siliconized 21 gauge butterfly needle, to cause minimum disruption to the vein while taking multiple blood samples. To minimize activation of the hemostatic system, a maximum of three venepunctures was specified. The cannula remained in place over the entire study time period, and venous blood samples of ˜30 ml were withdrawn at each sampling timepoint, discarding the first 2 mL on each occasion. After blood sample collection, the cannula was flushed with saline to prevent blockage. For measurements of platelet function and clotting time, blood was collected into plastic syringes and transferred into citrated blood collection tubes (final concentration sodium citrate, 13 mmol/L). For measurement of C-reactive protein (CRP), a single baseline blood sample (5 mL) was taken into EDTA anticoagulant (final concentration, 1.6 g/L). For measurement of fibrinopeptide A at each timepoint, 4.5 mL blood was collected into 0.5 mL of a mixed anticoagulant containing EDTA, trasylol and chloromethylketone. Blood samples were incubated at 37° C. in a portable incubator for transfer to the laboratory. Any blood samples showing evidence of activation, defined as a fibrinopeptide A concentration higher than 6 μg/L, were discarded. Any volunteers showing evidence of an elevated inflammatory response, as evidenced by a baseline C-reactive protein concentration higher than 6 mg/L, were withdrawn from the study for the period affected, and the scheduled intervention was undertaken at a later date.

### 3.1.5 Ex Vivo Platelet Aggregation Studies

Measurement of the extent of ADP and collagen-induced platelet aggregation in platelet-rich plasma was carried out at each timepoint. Different agonist concentrations may be used to approximate different physiological conditions. In order to collect data under conditions of suboptimal platelet stimulation, a standardized lower concentration (3 μmol/L for ADP, 3 mg/L for collagen) was defined as suboptimal, while a standardised upper concentration (7.5 μmol/L for ADP, 5 mg/L for collagen) was defined as optimal. These agonist concentrations were used for all measurements. Effects on platelet aggregation observed after treatment or control interventions are expressed as the percentage change in area under the aggregation curve after consumption of extract/ placebo, compared to baseline values.

### 3.1.6 Supplementary Measurements

Detection of high plasma CRP was carried out using a semi-quantitative latex agglutination assay (Dade Behring, UK), which detected levels in plasma >6 mg/L. This threshold is taken as an indication of acute inflammatory system activation, such as may be associated with infection (e.g. onset of a viral infection or a cold) or injury (e.g. tendonitis). Samples displaying signs of such acute activation should not be used for platelet function studies.

Measurement of FPA was carried out by ELISA (Zymutest FPA assay, HyphenBioMed, France), on plasma from which fibrinogen had been removed by bentonite adsorption treatments. Presence of FPA in plasma at levels greater than 6 μg/L was taken as an indication of haemostatic system activation during blood sampling. Such samples should not be used for platelet function measurement as results obtained will not be reliable. Thus circulating CRP levels and blood sample FPA levels were used to indicate suitability of samples for platelet measurements.

### 3.2 Results

No blood samples drawn during this study displayed levels of circulating CRP higher than the threshold 6 mg/L, indicating that acute phase activation was not present in any subject during the study sampling days. Similarly, in blood samples drawn for this study, no samples showed FPA levels higher than the threshold 6 μg/L. Thus all blood samples received were judged suitable for platelet function studies. This screening data is not included.

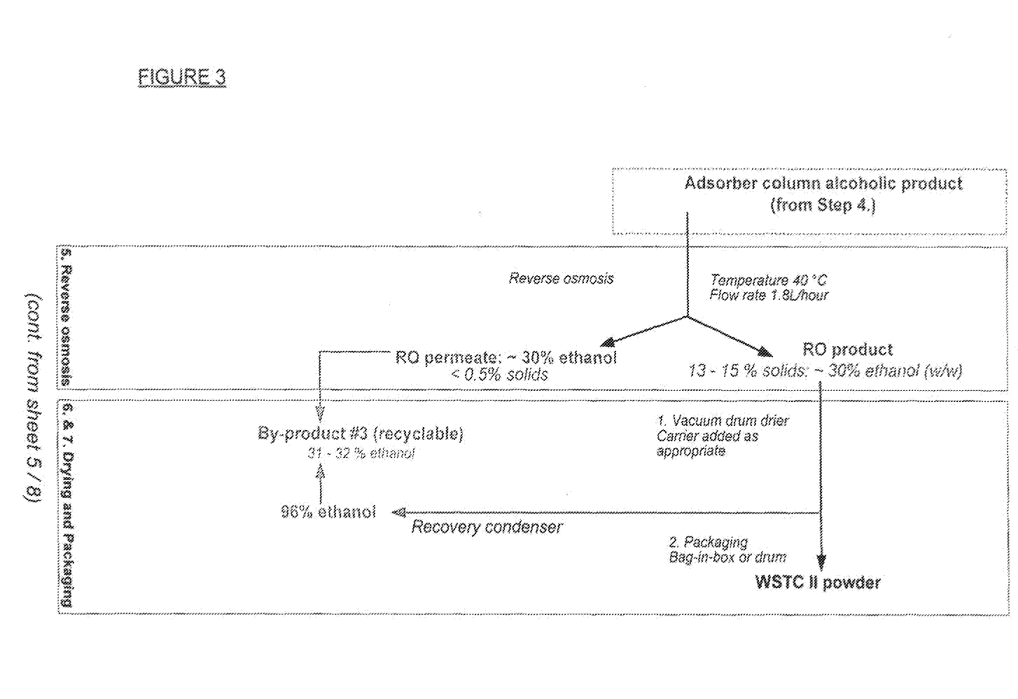

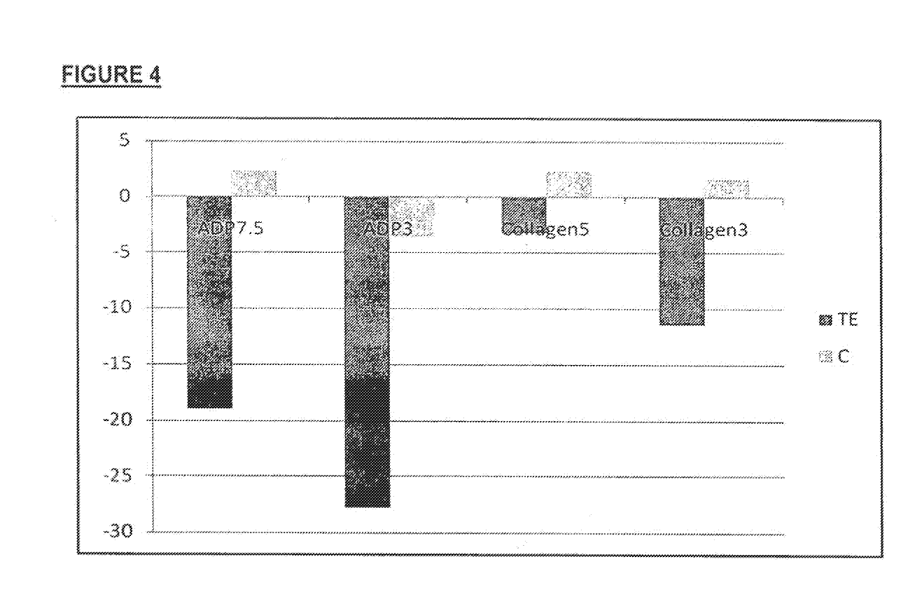

The data presented in FIG. 4 illustrate platelet aggregation measurements carried out at baseline (t=0) and at 3 hours post-consumption of treatment supplements (t=3). Results are expressed as % inhibition of platelet aggregability, compared to baseline values. The figure demonstrates that tomato extracts enriched in phenolic bioactives cause a reduction from baseline platelet aggregation of between 18% and 28% for ADP mediated aggregation, and between 3% and 12% for collagen mediated aggregation, 3 hours after consumption. Consumption of the control supplement resulted in a change from baseline aggregation of between 2% and 4% for ADP-mediated aggregation, and approximately 2% for collagen-mediated aggregation, after 3 hours. The differences between baseline and 3-hour time points were not significant for the control supplement, but were significant at P <0.001 for the tomato extract supplement. Differences between the control and tomato extract supplements were also significant at the P <0.001 level.

These results clearly demonstrate that tomato extracts enriched in phenolics are useful for treating conditions characterised by inappropriate platelet aggregation.

### EXAMPLE 4

The inventors prepared a number of products that represent preferred formulations comprising phenolic bioactives according to the invention.

### Yoghurt Drinks Containing Tomato Extracts

The tomato extracts prepared as described in FIGS. 2 and 3, or alternatively mixtures of individual bioactive phenolic/flavonoid derivatives obtained by synthesis, isolation or other means as described earlier, are suitable for incorporation into a yoghurt drink. An example of such a drink may be prepared as follows.

Drinking yoghurt, formulated without live probiotic cultures, should be pre-pasteurised and cooled to 4-8° C. The cooled yoghurt should be mixed with tomato extract as prepared in FIG. 2 in the ratio 50:1, or with tomato extract as prepared in FIG. 3 in the ratio 1000:1 (w/w). Alternatively, a mixture of Compounds 1, 5, 9, 18, 23 and 30 may be prepared by mixing 1.709 g, 9.576 g, 0.398 g, 0.727 g, 0.270 g and 0.18 g of the respective compounds, and this mixture may be combined with the cooled yoghurt in the ratio 500:1. Acidity should be checked and regulated with citric acid, and flavouring should be adjusted. If a probiotic culture is required in the final product, this should be added after adjustment, and the final mixture should be packaged into single-serve 150 g bottles.

Each single-serve 150 g bottle should then contain either 3 g tomato extract prepared according to FIG. 2, or 150 mg tomato extract prepared according to FIG. 3, or a total of 12.788 mg of the mixture of individual compounds described above. This represents a single daily dose. The final products should be stored at 4° C. for their recommended shelf life (typically between 14 and 21 days).

### Fat Spreads Containing Tomato Extract

Tomato extract prepared as described in FIG. 3, or further processed to give an encapsulate, is suitable for incorporation into fat spreads. An example of such a formulation may be produced by post-pasteurisation dosing the powdered, low-sugar tomato extract into pre-formulated, pasteurised and cooled fat spread In the ratio 200:1 (w/w). Alternatively, a mixture of Compounds 1, 5, 9, 18, 23 and 30 may be prepared by mixing 1.709 g, 9.576 g, 0.398 g, 0.727 g, 0.270 g and 0.108 g of the respective compounds, and this mixture may be combined with the cooled yoghurt in the ratio 2300:1. The mixture should be homogenised at high shear to ensure homogenous distribution, and packaged into multi-serve containers.

Label text should include the information that the normal daily intake of fat spread should be approximately 30 g. Consumption of 30 g fat spread per day will result in a daily intake of approximately 150 mg tomato extract, or a total of 12.788 mg of the mixture of individual compounds described, which constitutes a single daily dose. The spread should be stored at 4° C. for the duration of its shelf life (typically 90 days).

### Fruit Juice-Baaed Drinks Containing Tomato Extracts

The tomato extracts prepared as described in FIGS. 2 and 3 are both suitable for incorporation into a fruit-juice based drinks. An example of such a drink may be prepared as follows.

Dilute orange juice concentrate with water in the ratio 1:5.4. To the reconstituted juice, add 0.1% grapefruit flavour, 0.05% pineapple flavour, and 1.2% tomato extract as produced in FIG. 2, or alternatively, 0.01% of a mixture of Compounds 1, 5, 9, 18, 23 and 30 prepared by mixing 1.709 g, 9.576 g, 0.398 g, 0.727 g, 0.270 g and 0.108 g of the respective compounds.

Test acidity and sweetness, and add up to 5% citric acid (acidity regulator) and up to 2% sucralose, as required. Pasteurise for 90 seconds at 121° C.

Package the pasteurised mixture in 1 L cartons, or in single-serve cartons or bottles. 250 mL of the final drink as described should contain approximately 3 g tomato extract, or a total of 12.788 mg of the mixture of individual compounds described, equivalent to a single daily dose. Label details should contain this information and the advice to drink one 250 mL portion per day.

Other fruit juice concentrates are equally appropriate for use; alternatively fresh fruit juices, mixtures of fruit and vegetable juices, or mixtures containing variable amounts of pulp, may be prepared.

### Encapsulates

Prepare a 50% w/w solution of powdered, low-sugar tomato extract which has been manufactured as described in FIG. 3. Raise the temperature to 60° C. Mix with an equal volume of either: a melted and emulsified mixture of high-melting fats, e.g. triglycerides; a solution of dispersed polysaccharides, e.g. pectins, agars; or other suitable polymers. Homogenise with care to ensure correct blending. Produce an encapsulate using a technique such as temperature-controlled spray-drying, controlling particle size so that final particle size is <200μ. Additives such as colours, preservatives or free-flow agents may be added to the dispersion prior to spray drying, as appropriate.

The resulting encapsulate should contain between 12% and 20% tomato extract on a w/w basis. The encapsulate should be stored at <4° C., in the dark, in sealed foil wrapping materials. Dosage of the encapsulate should be in the range 400 mg-700 mg per day, when incorporated into food products.

### Sacheted Ready-to-Dissolve Formulations

The tomato extract as described in FIG. 3 is suitable for incorporation into pre-mixed, ready-to-dissolve single serving sachet formulations. An example of such a formulation may be prepared by mixing: 150 mg powdered, low-sugar tomato extract; 285 g maltodextrin; 6.5 g strawberry cream flavour; 0.8 g sucralose; 3.8 g citric add; 2.5 g natural beet red colour; and 0.25 g caramel. Alternatively, a mixture of Compounds 1, 5, 9, 18, 23 and 30 may be prepared by mixing 1.709 g, 9.576 g, 0.398 g, 0.727 g, 0.270 g and 0.108 g of the respective compounds, and 12.788 mg of this mixture added in place of tomato extract. The resulting ˜300 g dry powder mix can be presented in a single-serve foil-backed sachet, suitable for dissolving in between 50 mL and 300 mL water, to taste. Each 300 g mixture contains a single daily dose of tomato extract.

The powdered, sacheted formulation should be stored at room temperature, and presented with instructions to consume one sachet per day in water.

### Tablets

The tomato extract as described in FIG. 2 may used to prepare tablets for pharmaceutical or dietary supplement use, e.g. tabletting by direct compression, as follows.

The tomato extract should be milled/ground to a particle size range of 1-3μ prior to tabletting. The pre-ground powdered extract should be dry-blended with an excipient such as microcrystalline cellulose, or maltodextrin M700, to provide lubrication during the compression process. A ratio of 40% extract to 80% excipient is suitable, but ratios from 10:90 to 60:40 may also be used. Powdered colourants may also be added as required.

Using a conventional tabletting machine, set at a pressure of 1.5-2.0 tonnes/square inch, 212 g tablets of 5 kg hardness may be produced. Such tablets will contain 85 mg tomato extract per tablet. Storage in laminated aluminium foil blister packs is recommended. In such packaging, tablets will be stable to storage under temperatures up to 45° C. Two tablets should be taken together, once or twice per day, to achieve a recommended dosage level.

### EXAMPLE 5

In Example 3 the direct antiplatelet effects of a composition prepared according to the methods described in Example 2 were described. To illustrate that these antiplatelet effects are of a magnitude to affect blood fluidity or blood flow, further work was undertaken in which the effects of this composition on overall primary haemostasis was measured. Haemostasis, that is, the halting of bleeding by the clotting process, occurs in two parts. Primary haemostasis refers to the ability of whole blood to form platelet micro- and macro-aggregates under flowing conditions, and form an initial platelet clot on a collagen-rich surface (normally a blood vessel wall). Secondary haemostasis refers to the formation of a fibrin network in this primary clot, induced by thrombin, which leads to a more permanent clot which takes significant time to dissolve via fibrinolysis. Measurement of primary haemostasis gives data that may be more physiologically relevant than aggregation data alone, when examining the efficacy of the tomato extract composition in affecting blood fluidity and thus blood flow.

In the following Example, an experiment is described in which the effect on overall primary haemostasis of a composition prepared according to the methods described in Example 2 was tested, using a Platelet Function Analyser, the PFA-100®. The platelet function analyzer device has become a useful tool for measurement of primary hemostasis in small samples of blood. This test system is a microprocessor controlled instrument which emulates m vitro the platelet dependent phase of primary hemostasis, while delimiting the role of the rheological factors. Basically, the system monitors platelet interaction on collagen-ADP (COL-ADP) or collagen-epinephrine (COL-EPI) coated membranes. Samples of citrated blood are aspirated under controlled flow conditions (shear rate: 4,000-5,000/s) through a 150 micrometer aperture cut into the membrane. During the process, the growing platelet plug progressively blocks the blood flow through the aperture cut. The platelet hemostatic capacity in the blood sample is indicated by the time required for the platelet plug to occlude the aperture (closure time), which is expressed in seconds.

### 5.1 Study Protocol

### 5.1.1 Study Objectives and Short Outline

This study examined the ex vivo effect of consuming 3 g of tomato extract syrup (prepared according to the methods described in FIG. 2), compared to a control supplement, on primary haemostasis in healthy subjects.

### 5.1.2 Study Design

6 healthy adults aged 45-75 years, with normal hemostatic parameters (blood counts), no medical history of serious disease or hemostatic disorders, and not consuming dietary supplements or drugs known to affect platelet function, were recruited. Written informed consent was obtained, and the study was approved by Grampian Research Ethics Committee. Baseline blood samples (anticoagulated with acid citrate dextrose buffer) were taken from fasted subjects between 07:00 and 08:30. Directly after collection of the baseline sample, subjects consumed either a treatment (TE) or a control supplement. Further blood samples were then taken at time t=3 hours, and t=5 hours after supplementation.

### 5.1.5 Ex Vivo Measurement of Primary Haemostasis

Measurement of PFA-100 closure time in whole blood samples was carried out at each timepoint. Measurements were carried out using collagen-epinephrine membranes. Briefly, cartridges containing the appropriate membranes were brought to room temperature, and 900 μl of anticoagulated whole blood was inserted into the reservoir of each cartridge. The cartridges were then immediately inserted into the processing unit of the PFA-100. The blood was aspirated automatically from the reservoir through the cartridge membrane at high shear, until the membrane aperture was closed (closure time) or for a maximum of 300 s in the event that no clot was formed. Closure times were recorded and a printout produced. All measurements were carried out a minimum of 30 minutes after blood sampling.

### 5.2 Results

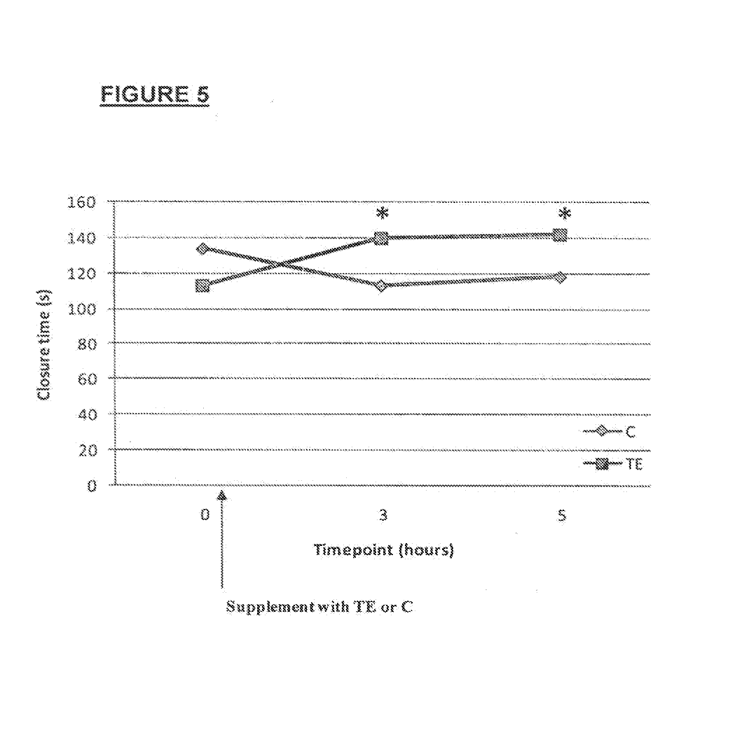

Average closure times for each treatment are presented graphically in FIG. 5. In this Figure recorded average closure times are shown for the baseline (time 0 relative to supplementation with treatment (TE) or control (C), and at 3 hours and 5 hours after supplementation with TE or C. n=3 for each group, and data were analysed by ANOVA. Significant differences between C and TE are indicated on the graph by * (P=0.011).

### 5.3 Conclusions

Results demonstrate that tomato extracts representing compositions according to the invention result in an average increase In PFA-100 closure time of 24% from baseline values, 3 and 5 hours after consumption. Consumption of the control supplement resulted in an average decrease from baseline closure times of 16% after 3 hours, and 12% after 5 hours. The differences between baseline and 3 and 5-hour time points were not significant for the control supplement, but were significant at P=0.011 for the tomato extract supplement. Differences between the control and tomato extract supplements were significant (P =0.011).

The results show that the tomato extract supplement compositions in accordance with the invention increase the time taken for a platelet clot to form in each cartridge aperture, implying that the platelet hemostatic potential has decreased. The longer time required for a clot to form reflects a higher blood fluidity.

These results clearly demonstrate that compositions (such as tomato extracts) according to the invention are useful in reducing blood fluidity. This supports their use in normalising blood flow.

1. A composition comprising a therapeutically effective amount of a compound of general formula (I): 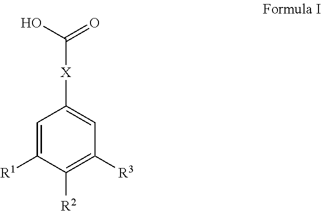
wherein R1, R2 and R3 are independently selected from H, OH and OMe; and
wherein X is C₁ or C₂ and wherein for C₂ each carbon is linked by a single or multiple bond and is substituted with one or more H or OH.

2. The composition according to claim 1 wherein the compound is cinnamic acid or a derivative thereof.

3. The composition according to claim 2 wherein the compound is a derivative of cinnamic acid selected from the group consisting of: p-Coumaric acid, Caffeic acid, Ferulic acid and Sinapic acid.

4. The composition according to claim 1 wherein the compound is benzoic acid or a derivative thereof.

5. The composition according to claim 4 wherein the compound is a derivative of benzoic acid selected from the group consisting of: p-Hydroxybenzoic acid, Protocatechuic acid, Gallic Acid, Vanillic acid and syringic acid.

6. The composition according to claim 1 wherein the compound is conjugated with a sugar.

7. The composition according to claim 6 wherein the sugar is 1-5 hexose or pentose sugar residues.

8. The composition according to claim 6 wherein the compound is selected from the group consisting of: Caffeic acid 3-O-glycoside, Caffeic acid 4-O-glycoside, Ferulic acid 4-O-glycoside, and p-Coumaric acid 4-O-glycoside.

9. The composition according to claim 1 wherein the compound is conjugated with another molecule by an ester bond.

10. The composition according to claim 9 wherein the compound is selected from the group consisting of Caffeoylquinic acids, 3-O-Caffeoylquinic acid, 4-O-Caffeoylquinic acid, 5-O-Caffeoylquinic acid, Feruloylquinic acids, p-Coumaroylquinic acids, Caffeoyltartaric acids, Feruloyltartaric acids, p-Coumaroyltartaric acids and dimers of quinic acid derivatives.

11. The composition according to claim 1 further comprising a flavonoid with activity for treating or preventing the development of medical conditions characterised by inappropriate platelet aggregation.

12. The composition according to claim 1 further comprising a nucleoside or nucleotide with activity for treating or preventing the development of medical conditions characterised by inappropriate platelet aggregation.

13. (canceled)

14. A nutritional product comprising a therapeutically effective amount of the composition as defined by claim 1.

15. A pharmaceutical product comprising a therapeutically effective amount of the composition as defined by claim 1.

16. A method of treating or reducing the likelihood of the development of medical condition in a subject characterised by inappropriate platelet aggregation, comprising:
providing a composition as defined by claim 1; and
administering a therapeutically effective amount of the composition to the subject to treat or prevent the development of the medical condition.

17. The method according to claim 16, wherein the treatment or prevention of the medical condition characterised by inappropriate platelet aggregation is for a purpose selected from the group consisting of: maintaining heart health by reducing platelet aggregation; benefiting the circulation; and normalizing or otherwise benefiting blood flow.
